(19) United States
(12) Patent Application Publication
(10) Pub. No.: US 2024/0377291 A1
(43) Pub. Date: Nov. 14, 2024

(54) AZETIDINE SUBSTITUTED ROSAMINES USEFUL FOR STAINING MITOCHONDRIA

(71) Applicant: TOCRIS COOKSON LIMITED, Bristol (GB)

(72) Inventors: Hannah Jane Maple, Bristol (GB); Paul Wood, Bristol (GB); Darcey Miller, Centennial, CO (US)

(21) Appl. No.: 18/686,700

(22) PCT Filed: Aug. 25, 2022
(86) PCT No.: PCT/GB2022/052188

§ 371 Date: Feb. 26, 2024

(30) Foreign Application Priority Data
Aug. 26, 2021 (GB) 2112198.3

(51) Int. Cl. G01N 1/30; C07D 405/14; C07F 7/08; G01N 33/50

(52) U.S. Cl. CPC G01N 1/30; C07D 405/14; C07F 7/0816; G01N 33/5005; G01N 2001/302

(57) ABSTRACT

Azetidine substitute rosamines useful for staining mitochondria of formula (I) are disclosed, wherein at least one of Y and Z is a substituted or unsubstituted azetidine group; X is selected from O, S, SO₂, Se, NR₁₂, P(O)R₁₂, CR₁₃R₁₄, SiR₁₃R₁₄, Te, and GeR₁₃R₁₄, and there is at least one Q group on the pendant phenyl, the Q group comprising a group selected from halo, maleimidyl, OSO₂R₁₃ and epoxide. Also disclosed are methods for staining mitochondria involving incubating a sample in a composition comprising the compound, and analysing mitochondria, involving staining a sample of mitochondria, optionally fixing the cells, illuminating the stained sample using light of an appropriate wavelength to fluoresce the compound, and observing or imaging a magnified image of the sample.

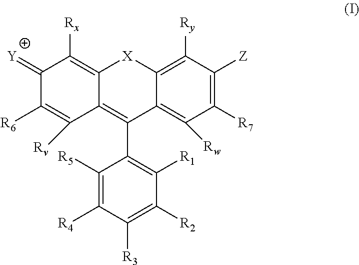

### FIELD OF THE INVENTION

The present invention relates to compounds useful for staining mitochondria, to compositions comprising such compounds, to methods of staining or analysing mitochondria, and to methods of detecting mitochondrial conditions.

### BACKGROUND

Functioning mitochondria underpin many critical cellular processes and mitochondrial dysfunction can therefore be a key factor in disease. Changes of mitochondrial shape, structure and function sometimes occur in response to changes in energy demand and cellular environment and in some animal (including human) diseases. Mitochondrial diseases may occur because of mutations (inherited or acquired), in mtDNA. Some diseases may also arise from the effects of drugs, infections or other causes.

It is helpful to directly image or observe mitochondria to further understand the nature and pathology of disease or to determine mitochondrial location and morphology for medical or research purposes.

Fluorescent dyes for selectively staining mitochondria are widely used in life sciences research, in applications such as fluorescence microscopy, flow cytometry and high-content screening. Most commercially available mitochondrial stains are organic fluorophores that accumulate in the mitochondrial matrix due to the transmembrane potential, for example MitoTracker™ dyes.

Fluorescent mitochondrial markers (or stains) should combine brightness with high photostability and low toxicity. Photostability is particularly important for studying live-cell mitochondrial morphology because mitochondria are dynamic, undergoing fusion and fission and it is desirable to be able to study this attribute over an extended time period without loss of signal or dye-induced toxicity. Overall brightness (typically measured as the product of the extinction coefficient and quantum yield) influences the concentration of stain that can be used and the final image quality. Increased brightness is a beneficial feature for mitochondrial markers. Dyes for use in imaging mitochondria also need to selectively accumulate in the mitochondria.

There is a need for improved stains that combine increased brightness and photostability with low toxicity and can successfully accumulate in both live and fixed cells. Further, it is desirable for certain applications to be able to fix cells prior to imaging whilst preserving specific mitochondrial staining.

It is an aim of the present invention to address this need.

### SUMMARY

In a first aspect, there is accordingly provided a compound comprising a cationic species of formula (I):

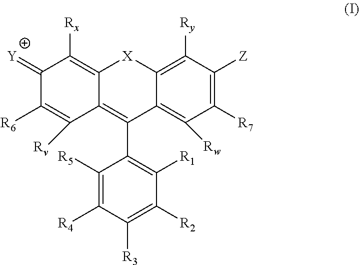

or a solvate, or tautomer thereof; and a counter ion;
wherein:
Y is a substituted or unsubstituted azetidine ring and Z is selected from OR₁₇ or a substituted or unsubstituted azetidine ring;
X is selected from O, S, SO₂, Se, NR₁₂, P(O)R₁₂, CR₁₃R₁₄, SiR₁₃R₁₄, Te, and GeR₁₃R₁₄;
R₁, R₂, R₃, R₄, and R₅ are each independently selected from H, C₁ to C₈ alkyl, OR₁₅, C(O)OR₁₆, NHC(O)R₁₅, C(O)NHR₁₅, halo, and a group of formula Q, wherein at least one of R₁, R₂, R₃, R₄, and R₅ is a group of formula Q;
Rᵥ, R_{w}, Rₓ, R_{y}, R₆, R₇ are each independently selected from H, C₁ to C₈ alkyl and halo;
R₁₆ is selected from C₁ to C₈ alkyl, optionally substituted aryl or optionally substituted heteroaryl;
R₁₇ is selected from H, C₁ to C₈ alkyl, optionally substituted aryl or optionally substituted heteroaryl; and
Q is a group comprising L-M_{A},
   wherein M_{A} is selected from —CR₁₈R₁₉M_{B}, NHC(O)CR₁₈R₁₉M_{B}, C(O)NHCR₁₈R₁₉M_{B}, NHC(O)-L-CR₁₈R₁₉M_{B}, —C(O)NH-L-CR₁₈R₁₉M_{B}, —O—CR₁₈R₁₉M_{B} and —O-L-CR₁₈R₁₉M_{B};
   each M_{B} is independently selected from halo, maleimidyl, OSO₂R₁₃, and

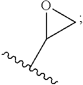 [SMILES: *C1CO1]

each L is an independently selected divalent linker group, optionally independently selected from C₁ to C₈ alkylene, substituted or unsubstituted phenylene or is absent, and
   R₁₈ and Rig; are each independently selected from H and CH₃; and
R₁₂, R₁₃, R₁₄, and R₁₅ are each independently selected from H, C₁ to C₈ alkyl, optionally substituted aryl or optionally substituted heteroaryl.

Such a compound according to formula (I) is greatly advantageous because it may be used to stain mitochondria and provides enhanced photostability with excellent brightness and is able to accumulate in mitochondria that are both live and fixed.

A cationic mitochondrial stain of formula (I) may optionally be generated by oxidation within mitochondria or intracellularly of a compound comprising an alternative, reduced form of formula (I), for example as shown in formula (Ib) below:

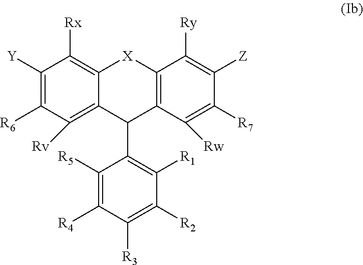

Suitably, at least one of Y and Z is a substituted or unsubstituted azetidine group of formula:

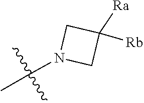

wherein R_{A} and R_{B} are independently selected from H, C₁ to C₈ alkyl, OR₂₀, C(O)OR₂₀, NHC(O)R₂₀, C(O)NHR₂₀, halo, NR₂₀R₂₁, —CN, —NC, optionally substituted aryl or optionally substituted heteroaryl; wherein R₂₀ and R₂₁ are independently selected from H, and C₁ to C₈ alkyl.

Preferably, R_{A} and R_{B} may be independently selected from H, halo, C₁ to C₈ alkyl, and OR₂₀. Halo may be F.

Thus, in some embodiments, Y or Z may be independently selected from:

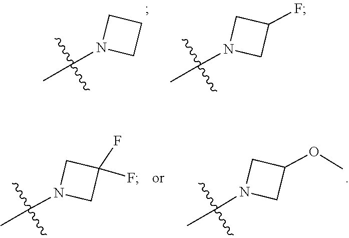

In some other embodiments, Y or Z may be independently selected from:

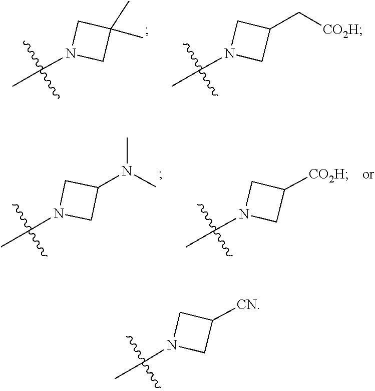

The compound comprises a cationic species of formula (I) and a counter ion. This is advantageous because such a cationic species may be a delocalized lipophilic cation and may selectively accumulate in mitochondria due to the negative potential gradient produced by the mitochondrial membrane.

Thus, suitably such a cationic species may be of formula (II):

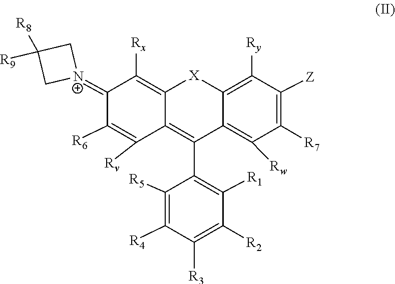

wherein R₈ and R₉ are independently selected from H, C₁ to C₈ alkyl, OR₂₀, C(O)OR₂₀, NHC(O)R₂₀, C(O)NHR₂₀, halo, NR₂₀R₂₁, —CN, —NC, optionally substituted aryl or optionally substituted heteroaryl; wherein R₂₀ and R₂₁ are as defined above.

Preferably, R₈ and R₉ may be independently selected from H, halo, C₁ to C₈ alkyl, and OR₂₀. Halo may be F.

The counter ion will usually result from the method of synthesis of the cationic species. The counter ion may be changed using ion exchange or other methods as known in the art.

Suitably, the counter ion may be a biologically compatible counter ion. A biologically compatible counter ion is not toxic in use and does not have a substantially harmful effect on biomolecules.

The counter ion may be selected from halide, carboxylate, oxalate, trifluoroacetate, sulfate, alkanesulfonate, arylsulfonate, phosphate, perchlorate, tetrafluoroborate, tetraphenylboride, hexafluorophosphate, nitrate and anions of aromatic or aliphatic carboxylic acids. Suitably, the counter ion may be selected from chloro, acetate or trifluoroacetate.

Suitably, the cationic species may be of formula (III):

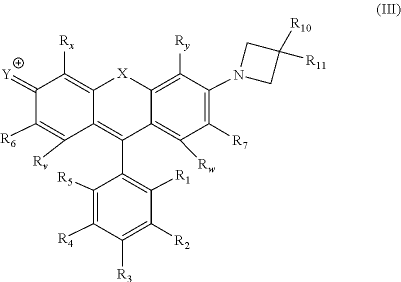

wherein R₁₀ and R₁₁ are independently selected from H, C₁ to C₈ alkyl, OR₂₀, C(O)OR₂₀, NHC(O)R₂₀, C(O)NHR₂₀, halo, NR₂₀R₂₁, —CN, —NC, optionally substituted aryl or optionally substituted heteroaryl; wherein R₂₀ and R₂₁ are as defined above.

Preferably, R₁₀ and R₁₁ may be independently selected from H, halo, C₁ to C₈ alkyl, and OR₂₀. Halo may be F.

Suitably, the cationic species may be of formula (IV):

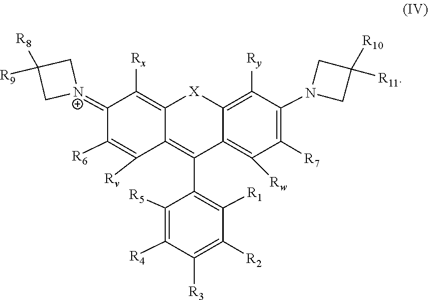

Structurally, the compound may comprise a species having an azetidine substituted rosamine (or rosamine analogue wherein X is O, S, SO₂, Se, NR₁₂, P(O)R₁₂, CR₁₃R₁₄, SiR₁₃R₁₄, Te, or GeR₁₃R₁₄) that may have halo, alkyl or other substituents on the pendant phenyl group and/or elsewhere.

Suitably, the Q group may be in the ortho position on the pendant phenyl ring. Thus, at least one of R₁ and R₅ may be Q.

Additionally or alternatively, the Q group may be at the para position. Thus, R₃ may be Q.

Additionally or alternatively, the Q group may be at one or both meta positions. Thus, at least one of R₂ and R₄ may be Q

Where present, one or more L groups may comprise an alkylene chain —(CH₂)ₘ—, wherein m is 1 to 8, suitably m may be 1 to 6. One or more L groups may be a substituted or unsubstituted phenylene group, for example in NHC(O)-L-CR₁₈R₁₉M_{B}, of formula (if R₁₈ and R₁₉ are each H):

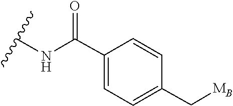

L may be absent.

The preferred M_{B} is halo, more preferably Cl.

Suitably, the compound may comprise a cationic species selected from species of formulae:

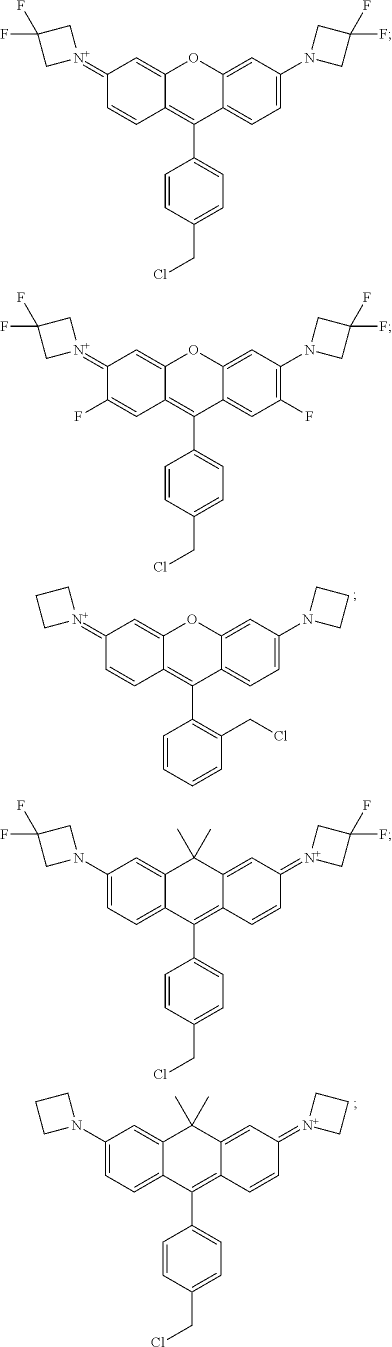 [SMILES: FC1(F)CN(c2ccc3c(-c4ccc(CCl)cc4)c4ccc(=[N+]5CC(F)(F)C5)cc-4oc3c2)C1 ; Fc1cc2c(-c3ccc(CCl)cc3)c3cc(F)c(=[N+]4CC(F)(F)C4)cc-3oc2cc1N1CC(F)(F)C1 ; ClCc1ccccc1-c1c2ccc(=[N+]3CCC3)cc-2oc2cc(N3CCC3)ccc12 ; CC1(C)C2=CC(=[N+]3CC(F)(F)C3)C=CC2=C(c2ccc(CCl)cc2)c2ccc(N3CC(F)(F)C3)cc21 ; CC1(C)C2=CC(=[N+]3CCC3)C=CC2=C(c2ccc(CCl)cc2)c2ccc(N3CCC3)cc21]

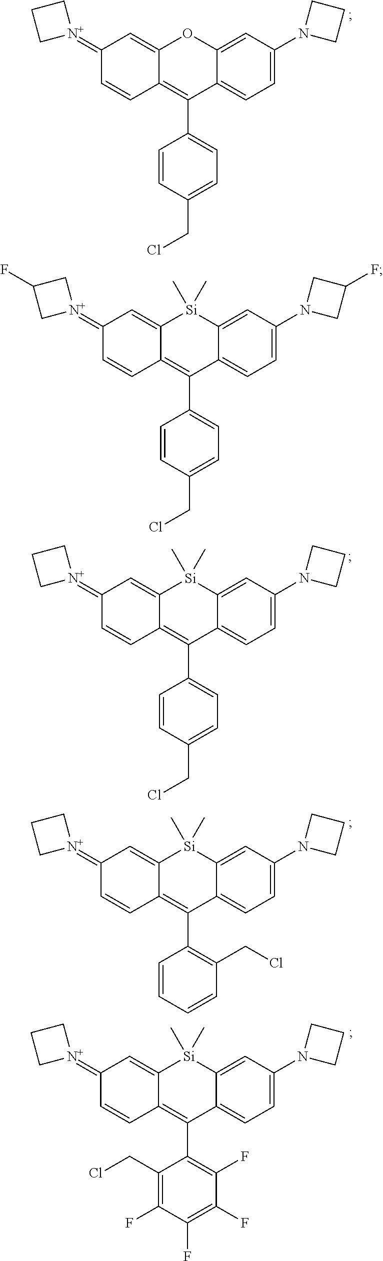 [SMILES: ClCc1ccc(-c2c3ccc(=[N+]4CCC4)cc-3oc3cc(N4CCC4)ccc23)cc1 ; C[Si]1(C)C2=CC(=[N+]3CC(F)C3)C=CC2=C(c2ccc(CCl)cc2)c2ccc(N3CC(F)C3)cc21 ; C[Si]1(C)C2=CC(=[N+]3CCC3)C=CC2=C(c2ccc(CCl)cc2)c2ccc(N3CCC3)cc21 ; C[Si]1(C)C2=CC(=[N+]3CCC3)C=CC2=C(c2ccccc2CCl)c2ccc(N3CCC3)cc21 ; C[Si]1(C)C2=CC(=[N+]3CCC3)C=CC2=C(c2c(F)c(F)c(F)c(F)c2CCl)c2ccc(N3CCC3)cc21]

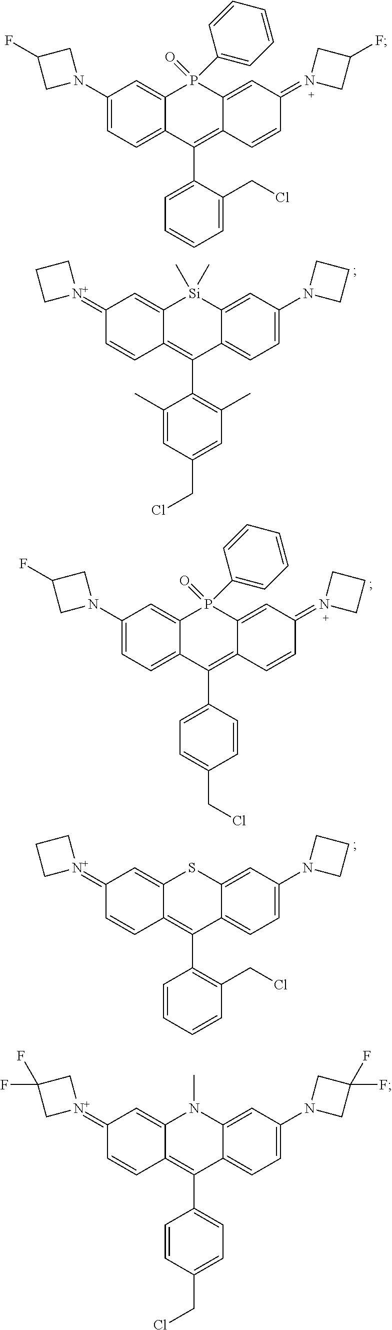

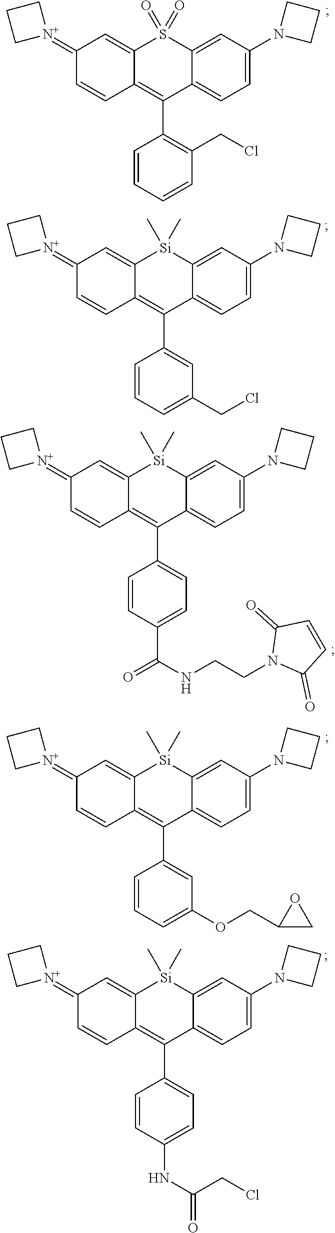 [SMILES: O=S1(=O)C2=CC(=[N+]3CCC3)C=CC2=C(c2ccccc2CCl)c2ccc(N3CCC3)cc21 ; C[Si]1(C)C2=CC(=[N+]3CCC3)C=CC2=C(c2cccc(CCl)c2)c2ccc(N3CCC3)cc21 ; C[Si]1(C)C2=CC(=[N+]3CCC3)C=CC2=C(c2ccc(C(=O)NCCN3C(=O)C=CC3=O)cc2)c2ccc(N3CCC3)cc21 ; C[Si]1(C)C2=CC(=[N+]3CCC3)C=CC2=C(c2cccc(OCC3CO3)c2)c2ccc(N3CCC3)cc21 ; C[Si]1(C)C2=CC(=[N+]3CCC3)C=CC2=C(c2ccc(NC(=O)CCl)cc2)c2ccc(N3CCC3)cc21]

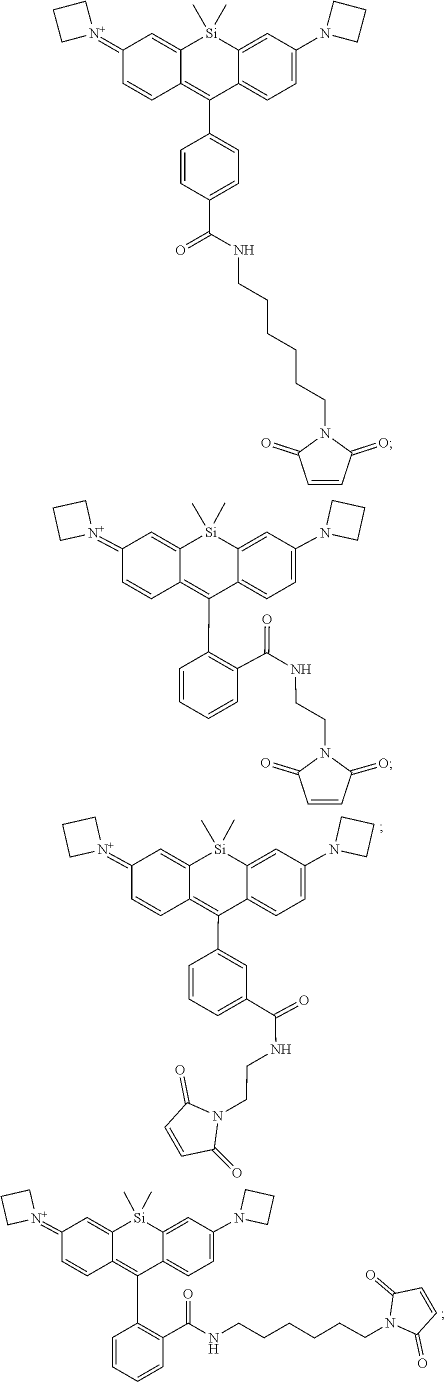 [SMILES: C[Si]1(C)C2=CC(=[N+]3CCC3)C=CC2=C(c2ccc(C(=O)NCCCCCCN3C(=O)C=CC3=O)cc2)c2ccc(N3CCC3)cc21 ; C[Si]1(C)C2=CC(=[N+]3CCC3)C=CC2=C(c2ccccc2C(=O)NCCN2C(=O)C=CC2=O)c2ccc(N3CCC3)cc21 ; C[Si]1(C)C2=CC(=[N+]3CCC3)C=CC2=C(c2cccc(C(=O)NCCN3C(=O)C=CC3=O)c2)c2ccc(N3CCC3)cc21 ; C[Si]1(C)C2=CC(=[N+]3CCC3)C=CC2=C(c2ccccc2C(=O)NCCCCCCN2C(=O)C=CC2=O)c2ccc(N3CCC3)cc21]

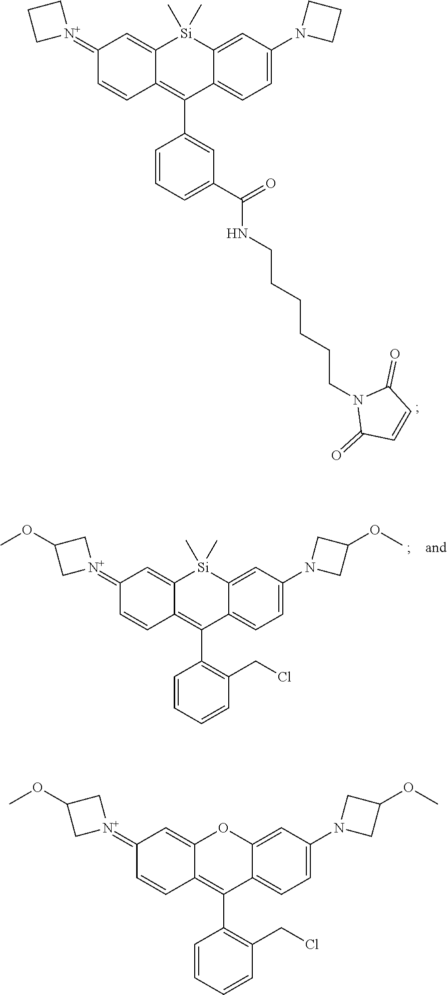

or solvates, or tautomers thereof; and a counter ion.

The compound may be isotopically labelled. For example, one or more hydrogens may be replaced with deuterium or tritium, or one or more carbons may be replaced with C-13.

In a second aspect, there is provided a composition for staining mitochondria, the composition comprising a compound as in the first aspect.

The composition may further comprise at least one organic solvent. The at least one organic solvent may be selected from DMSO, acetone, dimethylformamide, acetonitrile, dioxane, and THF.

The concentration of the compound in the composition may be in the range 10 nM to 1 μM, preferably 10 nM to 300 nM.

In a third aspect, there is provided a method for staining mitochondria, the method comprising: providing a sample containing mitochondria, and incubating the sample in a composition comprising a compound as in the first aspect.

Usually, incubating the sample is for a predetermined time, optionally in the range 10 mins to 2 hours and at a predetermined temperature, optionally in the range 20° C. to 39° C.

Usually, the sample containing mitochondria comprises a tissue sample. The sample containing mitochondria may be a plant, animal or fungal tissue sample, a sample of plant, animal or fungal cells or isolated plant, animal or fungal mitochondria. Examples of tissue samples include tissue sections, biopsy, blood draws, cytology samples, etc.

The sample containing mitochondria may comprise a sample containing fixed mitochondria and/or a sample containing mitochondria in fixed cells.

In a fourth aspect, there is provided a method of analysing mitochondria, the method comprising: staining a sample of mitochondria using a compound as in the first aspect, optionally fixing the cells, illuminating the stained sample using light of an appropriate wavelength to fluoresce the compound, and observing or imaging a magnified image of the sample.

The appropriate wavelength may be in the range 400 nm to 800 nm.

In a fifth aspect, there is provided a method of detecting or diagnosing a mitochondrial condition comprising staining a sample of mitochondria as in the third aspect and/or analysing a sample of mitochondria as in the fourth aspect.

Further particular and preferred aspects are set out in the accompanying independent and dependent claims. Features of the dependent claims may be combined with features of the independent claims as appropriate, and in combinations other than those explicitly set out in the claims, as supported by the description.

### Definitions

“Substituted,” when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

“Optionally substituted” refers to a parent group which may be un-substituted or which may be substituted with one or more substituents. Suitably, unless otherwise specified, when optional substituents are present the optional substituted parent group comprises from one to three optional substituents thus the group may be substituted with 0, 1, 2 or 3 of the optional substituents. Suitably, the group is substituted with 1, 2 or 3 of the optional substituents.

Optional substituents may be selected from C₁₋₈ alkyl, C₁₋₆ alkyl, C₂₋₇ alkenyl, C₂₋₇ alkynyl, C₁₋₁₂ alkoxy, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups. In some aspects, the optional substituents are 1, 2 or 3 optional substituents independently selected from OH, C₁₋₈ alkyl, C₁₋₆ alkyl, OC₁₋₁₂ alkyl, and halogen. More suitably, the optional substituents are selected from OH, C₁₋₈ alkyl and OC₁₋₁₂ alkyl; more suitably, the optional substituents are selected from C₁₋₈ alkyl and OC₁₋₁₂ alkyl.

“Independently” or “Independently selected” is used in the context of statement that, for example, “each R₁₆, R₁₇ is independently H, C₁₋₈ alkyl . . . ” and means that each instance of the functional group, e.g., R₁₆, is selected from the listed options independently of any other instance of R₁₆ or R₁₇ in the compound. Hence, for example, H may be selected for the first instance of R₁₆ in the compound; methyl may be selected for the next instance of R₁₆ in the compound; and ethyl may be selected for the first instance of R₁₇ in the compound.

C₁₋₈ alkyl: refers to straight chain and branched saturated hydrocarbon groups, having from 1 to 8 carbon atoms, and C₁₋₆ alkyl to straight chain and branched saturated hydrocarbon groups, having from 1 to 6 carbon atoms. Suitably a C₁₋₇ alkyl; suitably a C₁₋₆ alkyl; suitably a C₁₋₅ alkyl; more suitably a C₁₋₄ alkyl; more suitably a C₁₋₃ alkyl. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, n-octyl and the like.

“Alkylene” refers to a divalent radical derived from an alkane which may be a straight chain or branched, as exemplified by —CH₂CH₂CH₂CH₂—. The alkylene may have the number of carbons as discussed above for alkyl groups.

“Aryl” refers to fully unsaturated monocyclic, bicyclic and polycyclic aromatic hydrocarbons having at least one aromatic ring. Aryl groups as used herein preferably are preferably “C₅₋₂₀ Aryl” a fully unsaturated monocyclic, bicyclic and polycyclic aromatic hydrocarbons having at least one aromatic ring and having a specified number of carbon atoms that comprise their ring members (e.g., C₅₋₂₀ aryl refers to an aryl group having from 5 to 20 carbon atoms as ring members). The aryl group may be attached to a parent group or to a substrate at any ring atom and may include one or more non-hydrogen substituents unless such attachment or substitution would violate valence requirements. Suitably, a is selected from a C₆₋₁₂ aryl, more suitably, a C₆₋₁₀ aryl. Examples of aryl groups include phenyl.

“Halogen” or “halo”: refers to a group selected from F, Cl, Br, and I. Preferably, the halogen or halo is F or Cl. In some aspects, preferably the halogen is F. In other aspects, suitably the halogen is Cl.

“Maleimidyl” refers to the univalent radical of maleimide of formula:

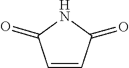

Attachment to other groups may be through C, or through N as indicated below:

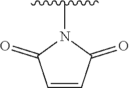 [SMILES: *N1C(=O)C=CC1=O]

where the wavy line indicate the point of attachment.

“Heteroaryl” refers to unsaturated monocyclic or bicyclic aromatic groups. Preferably heteroaryl is “C₅₋₁₀ heteroaryl” or “5- to 10-membered heteroaryl” an unsaturated monocyclic or bicyclic aromatic group comprising from 5 to 10 ring atoms, whether carbon or heteroatoms, of which from 1 to 5 are ring heteroatoms. Suitably, any monocyclic heteroaryl ring has from 5 to 6 ring atoms and from 1 to 3 ring heteroatoms. Suitably each ring heteroatom is independently selected from nitrogen, phosphorus, oxygen, sulfur and silicon.

The bicyclic rings include fused ring systems and, in particular, include bicyclic groups in which a monocyclic heterocycle comprising 5 ring atoms is fused to a benzene ring. The heteroaryl group may be attached to a parent group or to a substrate at any ring atom and may include one or more non-hydrogen substituents unless such attachment or substitution would violate valence requirements or result in a chemically unstable compound.

Examples of monocyclic heteroaryl groups include, but are not limited to, those derived from:
N₁: pyrrole, pyridine;
O₁: furan;
S₁: thiophene;
N₁O₁: oxazole, isoxazole, isoxazine;
N₂O₁: oxadiazole (e.g., 1-oxa-2,3-diazolyl, 1-oxa-2,4-diazolyl, 1-oxa-2,5-diazolyl, 1-oxa-3,4-diazolyl);
N₃O₁: oxatriazole;
N₁S₁: thiazole, isothiazole;
N₂: imidazole, pyrazole, pyridazine, pyrimidine, pyrazine;
N₃: triazole, triazine; and,
N₄: tetrazole.

Examples of heteroaryl groups which comprise fused rings, include, but are not limited to, those derived from:
O₁: benzofuran, isobenzofuran;
N₁: indole, isoindole, indolizine, isoindoline;
S₁: benzothiofuran;
N₁O₁: benzoxazole, benzisoxazole;
N₁S₁: benzothiazole;
N₂: benzimidazole, indazole;
O₂: benzodioxole;
N₂O₁: benzofurazan;
N₂S₁: benzothiadiazole;
N₃: benzotriazole; and
N₄: purine (e.g., adenine, guanine), pteridine;

As used herein, “solvate” refers to a complex of variable stoichiometry formed by a solute and a solvent. Solvates may be formed for crystalline compounds wherein solvent molecules are incorporated into the crystalline lattice during crystallization. The incorporated solvent molecules can be water molecules or non-aqueous molecules, such as but not limited to, ethanol, isopropanol, dimethyl sulfoxide, acetic acid, ethanolamine, and ethyl acetate molecules.

“Tautomer, “refers to a structural isomer of a compound that readily interconverts to another isomer.

“Fixed cells” refers to cells that have undergone a fixing process to substantially end biochemical reactions within the cells. References to “fixed mitochondria” refer to mitochondria that are or were present in cells that have undergone the fixing process or mitochondria that have undergone a fixing process in order to substantially end biochemical reactions within the mitochondria.

In this specification, “live mitochondria” refers to mitochondria that are functioning in the sense that there is a mitochondrial membrane potential and/or the membrane has not been substantially ruptured.

“Mitochondrial conditions,” as used herein are mitochondrial diseases or conditions involving or that may lead to mitochondrial dysfunction where mitochondria fail to produce enough energy for the body or parts of the body to function properly. Mitochondrial conditions may be chronic, and genetic. Mitochondrial dysfunction occurs when the mitochondria are affected by another disease or condition. Conditions that may lead to such mitochondrial dysfunction include Alzheimer's disease, muscular dystrophy, Lou Gehrig's disease, diabetes and cancer. Mitochondrial conditions/diseases include: Kearns-Sayre syndrome, Leber's hereditary optic neuropathy, Progressive external ophthalmoplegia, Mitochondrial encephalomyopathy with lactic acidosis and stroke-like episodes (MELAS), and Myoclonic epilepsy with ragged red fibres (MERRF).

The term “subject” as used herein refers to a human or non-human animal, suitably a mammal. Examples of non-human mammals include livestock animals such as sheep, horses, cows, pigs, goats, rabbits and deer; and companion animals such as cats, dogs, rodents, and horses.

As used herein the term “comprising” means “including at least in part” and is inclusive or open ended. When interpreting each statement in this specification that includes the term “comprising,” features, elements and/or steps other than that or those prefaced by the term may also be present. Related terms such as “comprise” and “comprises” are to be interpreted in the same manner. It should be understood that while various aspects in the specification are presented as “comprising,” this includes aspects that “consist essentially of” or “consist of” that aspect.

The term “consisting essentially of” limits the scope of a claim to the specified materials or steps “and those that do not materially affect the basic and novel characteristic(s)” of the claimed invention. When the phrase “consisting essentially of” appears in a clause of the body of a claim, rather than immediately following the preamble, it limits only the element set forth in that clause.

The term “consisting of” excludes any element, step, or ingredient not specified in the claim; “consisting of” defined as “closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

### BRIEF DESCRIPTION OF THE DRAWINGS

Embodiments of the present invention will now be described further, with reference to the accompanying drawings, in which:

FIG. 1 shows chemical structures of compounds according to the invention.

FIG. 2 shows images of antibody NBP2-23489 incubated on HeLa cells co-stained with COMPOUND 5 (left, far red mitochondrial marker) and antibody (middle) and COMPOUND 5 (right) separately stained.

FIG. 3 shows in A, a schematic representation of mitochondrial membrane de-polarisation experiments, and in B, images of the results.

FIG. 4 shows images of fixed COS-7 cells stained with COMPOUND 5 compared to a Comparator (MitoTracker Deep Red FM) at varying concentrations.

FIG. 5 shows images of fixed HeLa cells stained with COMPOUND 5 compared to a Comparator (MitoTracker Deep Red FM) at a single concentration.

FIG. 6 shows images of fixed HeLa cells stained with either COMPOUND 8 or COMPOUND 9 (both added before fixation). In each experiment, an antibody for the mitochondrial marker TOM20 has also been added. Co-localization of TOM20 and mitochondrial probes is directly compared in the merged data.

FIG. 7 shows images of fixed HeLa cells stained with COMPOUND 5 (added after fixation) or a Comparator (added before fixation). COMPOUND 5 and the Comparator have distinct absorption and emission maxima. Intensity profiles (lower panel) taken across a set frame are shown for both COMPOUND 5 and Comparator.

FIG. 8 shows normalized absorption and fluorescence emission spectra for COMPOUND 5 1-(7-(azetidin-1-yl)-10-(2-(chloromethyl)phenyl)-5,5-dimethyldibenzo[b,e]silin-3(5H)-ylidene)azetidin-1-ium chloride.

FIG. 9 shows normalized absorption and fluorescence emission spectra for COMPOUND 6 1-(7-(azetidin-1-yl)-10-(4-(chloromethyl)phenyl)-5,5-dimethyldibenzo[b,e]silin-3(5H)-ylidene)azetidin-1-ium chloride.

FIG. 10 shows normalized absorption and fluorescence emission spectra for COMPOUND 8 1-(6-(azetidin-1-yl)-9-(2-(chloromethyl)phenyl)-3H-xanthen-3-ylidene)azetidin-1-ium chloride.

FIG. 11 shows normalized absorption and fluorescence emission spectra for COMPOUND 9 1-(9-(2-(chloromethyl)phenyl)-6-(3-methoxyazetidin-1-yl)-3H-xanthen-3-ylidene)-3-methoxyazetidin-1-ium chloride.

### DESCRIPTION OF THE EMBODIMENTS

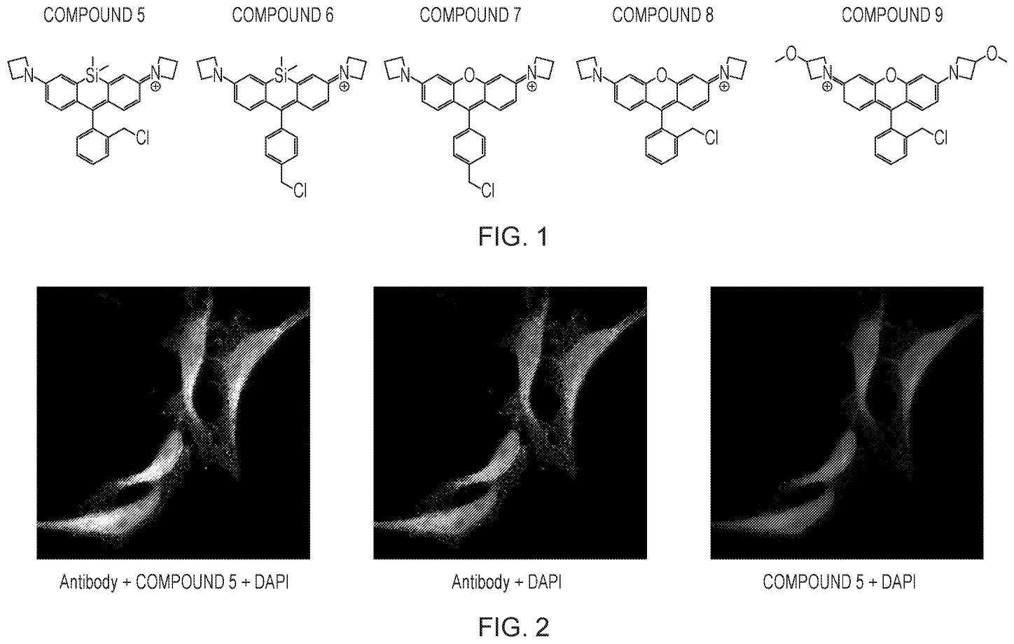

FIG. 1 shows the structures of compounds 5, 6, 7, 8 and 9 according to the invention. The compounds in FIG. 1 are azetidine-substituted rosamines and analogues, with ortho- or para-chloromethyl substituents. Compounds according to the invention are excellent for fixed-cell (and live-cell) fluorescent imaging of mitochondria.

The compounds outlined in FIG. 1 cover two core ‘series’ that are primarily defined by distinct excitation/emission profiles. Further compounds with cationic species as in formula I may have different excitation/emission wavelengths.

COMPOUND 5 is an excellent mitochondrial stain that localizes specifically to the mitochondria as demonstrated in FIG. 2. FIG. 2 shows images for a series of experiments where NBP2-23489 DRP1 Antibody (Novus Biologicals; Catalog #NBP2-23489) was co-stained with COMPOUND 5 (far red mitochondrial marker). NBP2-23489 was incubated on fixed, permeabilized HeLa cells at 8 μg/mL for 2 hours at room temperature. Cells were then incubated with an NL557 secondary antibody (R&D Systems; Catalog #NL007) for 1 hour at room temperature protected from light. Lastly, cells were incubated with COMPOUND 5 at 200 nM for 15 minutes at room temperature protected from light. Cells were cover-slipped with a DAPI-containing mounting media and imaged. Antibody staining was pseudo-coloured green, and mitochondrial marker staining was pseudo-coloured red. The images clearly show that antibody staining is localized to the mitochondria and cytoplasm and overlaps well with COMPOUND 5.

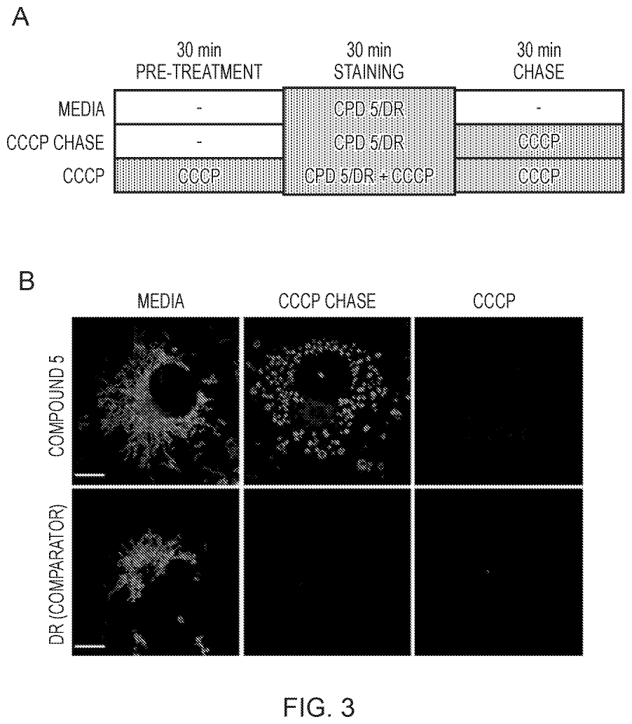

FIG. 3 demonstrates that COMPOUND 5 localizes to the mitochondria via the same mechanism as MitoTracker DeepRed (the Comparator), because de-polarizing the mitochondrial membrane with CCCP treatment prior to addition of the mitotrackers eliminates staining. Advantageously and surprisingly, COMPOUND 5 can stain the mitochondria more rapidly than the Comparator (MitoTracker deep red), since subsequent treatment with CCCP (“CCCP chase”) does not eliminate staining by COMPOUND 5.

In FIG. 3 (scale bar=10 μm), A) shows a schematic representation of the experimental setup. COS-7 cells were pre-treated with media or 20 μM CCCP for 30 minutes followed by a staining step with 100 nM COMPOUND 5 or 100 nM Comparator (MitoTracker Deep Red FM) for 30 minutes in the presence/absence of 20 μM CCCP. After the staining, excessive dye was washed out with media, with or without 20 μM CCCP for 30 minutes. B) shows depolarizing the mitochondrial membrane potential with CCCP reduces the mitochondrial accumulation of COMPOUND 5 or the Comparator (MitoTracker Deep Red FM). However, depolarizing the mitochondrial membrane potential for 30 minutes with CCCP after staining still results in a strong mitochondrial signal of COMPOUND 5, but not of the Comparator (MitoTracker Deep Red FM), see CCCP Chase. This suggests that COMPOUND 5 is still retained in mitochondria even after the loss of the mitochondrial membrane potential, whereas the mitochondrial accumulation of the Comparator (MitoTracker Deep Red FM) is reversible. In conclusion, mitochondrial accumulation of COMPOUND 5 and the Comparator (MitoTracker Deep Red FM) is membrane potential-dependent, but only reversible for the Comparator upon loss of the mitochondrial membrane potential.

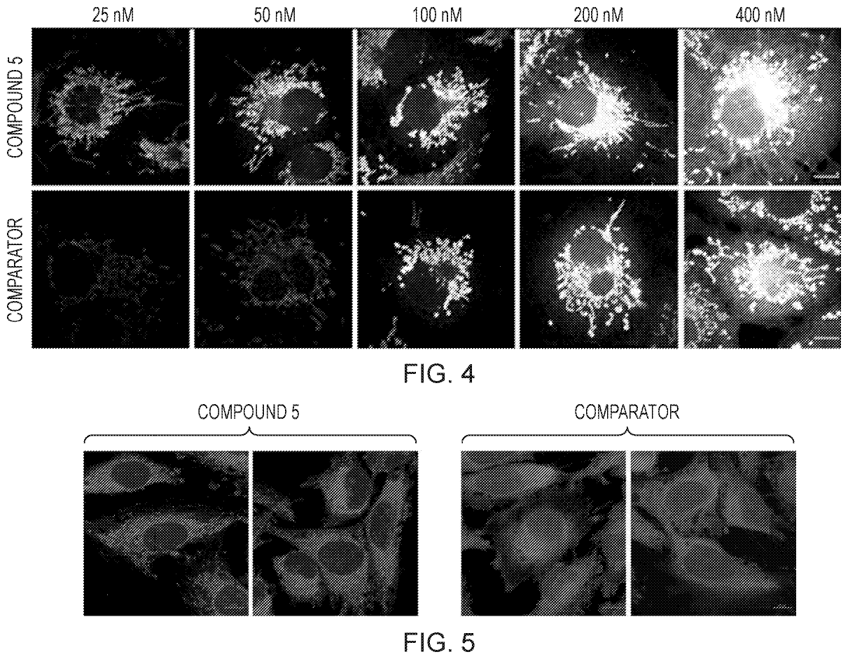

FIG. 4 demonstrates the improved performance of compounds of the invention compared to an existing commercial product (MitoTracker Deep Red). COMPOUND 5 shows significantly improved brightness compared to the Comparator, particularly at lower concentrations (25 nM and 50 nM). FIG. 4 shows optimization of concentrations required for imaging. Both COMPOUND 5 and the Comparator show some toxicity at 200 nM. COMPOUND 5 gives nice staining at 25-100 nM.

In FIG. 4 (scale bar=10 μm), images are shown after COS-7 cells were stained for 30 minutes with indicated concentrations followed by immediate fixation with 4% PFA for 20 minutes at 37° C. Images were acquired using a spinning disk confocal microscope with identical exposure time and laser power and were processed with identical settings to allow a direct comparison. Low concentrations of COMPOUND 5 (25-100 nM, 30 min) resulted in a good mitochondrial signal with little background and no obvious toxicity. Staining intensities for the Comparator decreased at low concentration. High concentration (above 200 nM) of COMPOUND 5 or the Comparator resulted in mitochondrial alterations (fragmentation and peri-nuclear clustering suggesting toxic effects). Moreover, both dyes stained additionally other cellular structures like the ER and the nuclear envelope at high concentrations.

FIG. 5 (scale bar=10 μm), demonstrates that COMPOUND 5 retains clearer staining of mitochondria following fixation, compared to a Comparator. In FIG. 5 (scale bar=10 μm), images are shown after HeLa cells were stained with 100 nM probe for 40 minutes, followed by fixation with 4% PFA for 10 minutes. Laser power and gain have been optimized for each individual probe in this figure.

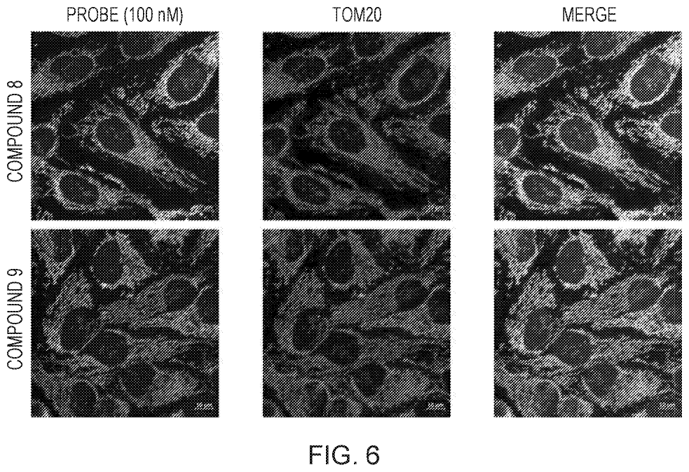

FIG. 6 (scale bar=10 μm), demonstrates that both COMPOUND 8 and COMPOUND 9 are selective mitochondrial stains that retain staining fidelity after fixation, since they both co-localize with the mitochondrial marker TOM20.

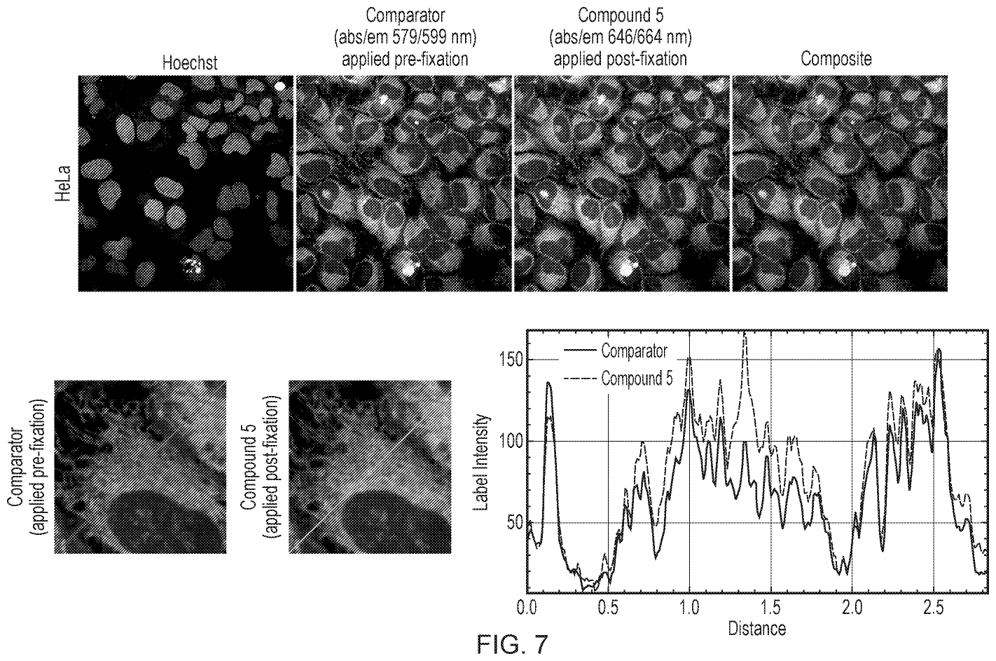

FIG. 7 shows images of fixed HeLa cells. Live HeLa cells were stained with a Comparator (250 nM, 45 min) and were then fixed (4% PFA, 20 min). After fixation, COMPOUND 5 (75 nM) was added and the cells were imaged. COMPOUND 5 and Comparator have distinct absorption and emission profiles and spill over between channels was not observed. Intensity profiles for both mitochondrial stains were taken across a set frame (lower panel) and compared. The data shows very high similarity in staining profile between the Comparator and COMPOUND 5, suggesting that COMPOUND 5 can be applied post-fixation.

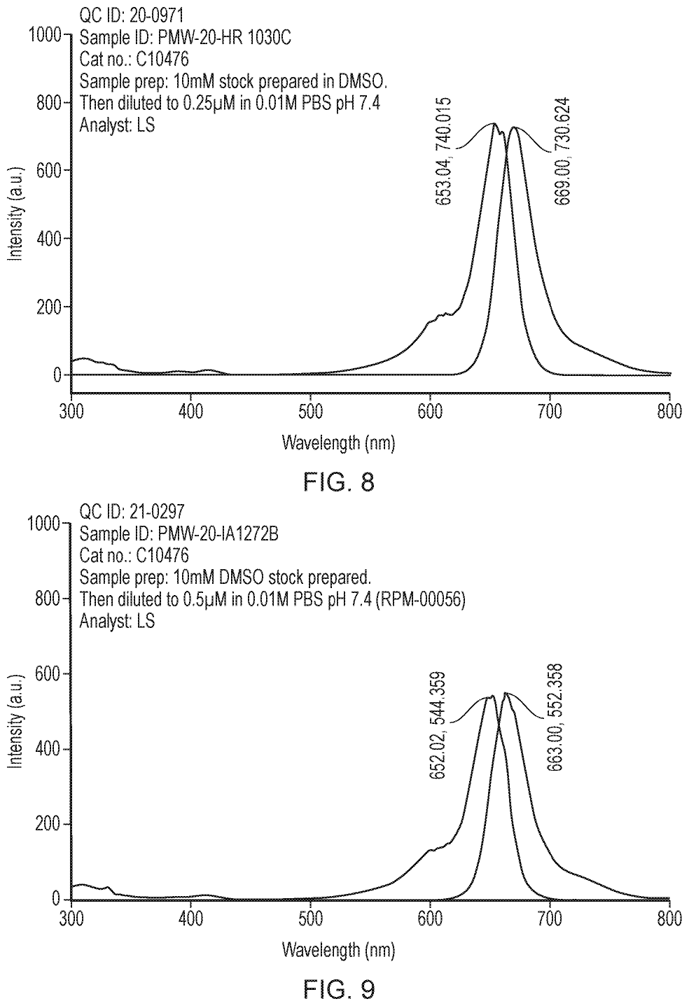

FIG. 8 shows normalised intensity (a.u) against wavelength for emission and absorption of COMPOUND 5 1-(7-(azetidin-1-yl)-10-(2-(chloromethyl)phenyl)-5,5-dimethyldibenzo[b,e]silin-3(5H)-ylidene)azetidin-1-ium chloride.

FIG. 9 shows normalised intensity (a.u) against wavelength for emission and absorption of COMPOUND 6 1-(7-(azetidin-1-yl)-10-(4-(chloromethyl)phenyl)-5,5-dimethyldibenzo[b,e]silin-3(5H)-ylidene)azetidin-1-ium chloride.

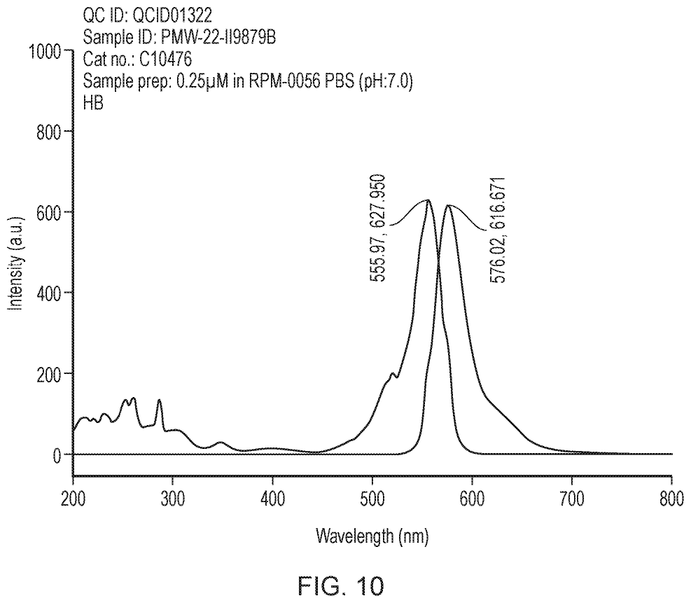

FIG. 10 shows normalised intensity (a.u) against wavelength for emission and absorption of COMPOUND 8 1-(6-(azetidin-1-yl)-9-(2-(chloromethyl)phenyl)-3H-xanthen-3-ylidene)azetidin-1-ium chloride.

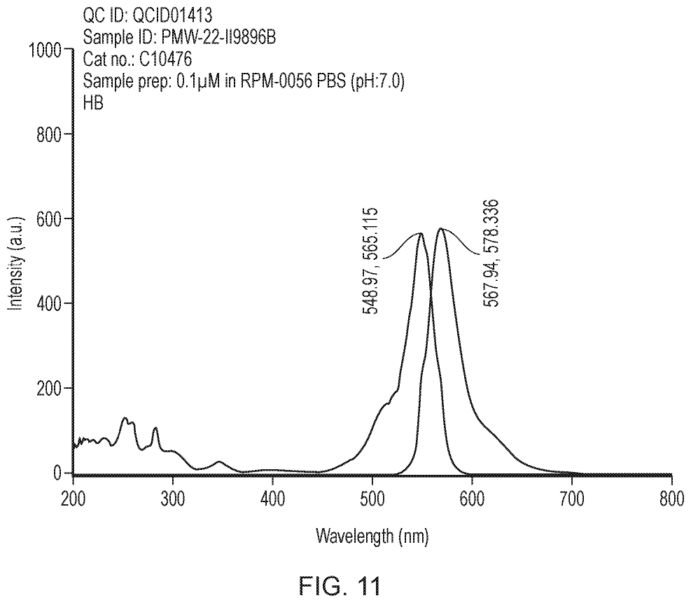

FIG. 11 shows normalised intensity (a.u) against wavelength for emission and absorption of COMPOUND 9 1-(9-(2-(chloromethyl)phenyl)-6-(3-methoxyazetidin-1-yl)-3H-xanthen-3-ylidene)-3-methoxyazetidin-1-ium chloride.

### General Chemistry Methods

All reagents and solvents were purchased from commercial sources and used without further purification. Nuclear magnetic resonance spectra were recorded on a Bruker Avance III HD spectrometer operating at 400 MHz for ¹H NMR and 100 MHz for ¹³C NMR. ¹H NMR and ¹³C NMR chemical shifts (5) are reported in parts per million (ppm) and are referenced to residual protium in solvent and to the carbon resonances of the residual solvent peak respectively.

Purification by flash chromatography was performed using pre-packed silica gel columns and either a Buchi Reveleris, a Biotage Isolera or a Biotage Selekt system. Analytical thin layer chromatography was performed on glass plates pre-coated with silica gel (Analtech, UNIPLATE™ 250 m/UV254), with visualization being achieved using UV light (254 nm) and/or by staining with alkaline potassium permanganate dip.

Reaction monitoring LC-MS analyses were conducted using Agilent InfinityLab LC/MSD systems. High resolution mass spectral (HRMS) data was collected using an Agilent 6545 LC/Q-TOF system.

Normalized absorption and fluorescence emission spectra were recorded in 10 mM PBS pH 7.3 at the concentration noted for each sample following dilution of a DMSO stock solution. Absorption spectra were recorded with an Agilent Cary 60 UV-Vis spectrophotometer using genuine precision quartz cells from Lovibond with a 1 cm path length. Fluorescence spectra were recorded on an Agilent Cary Eclipse Fluorescence Spectrophotometer using high precision Quartz Suprasil cells from Hellma Analytics and a 1 cm path length.

### EXAMPLES

The invention is further illustrated by the following Examples.

### Example 1—1-(7-(azetidin-1-yl)-10-(2-(chloromethyl)phenyl)-5,5-dimethyldibenzo[b,e]silin-3(5H)-ylidene)azetidin-1-ium chloride

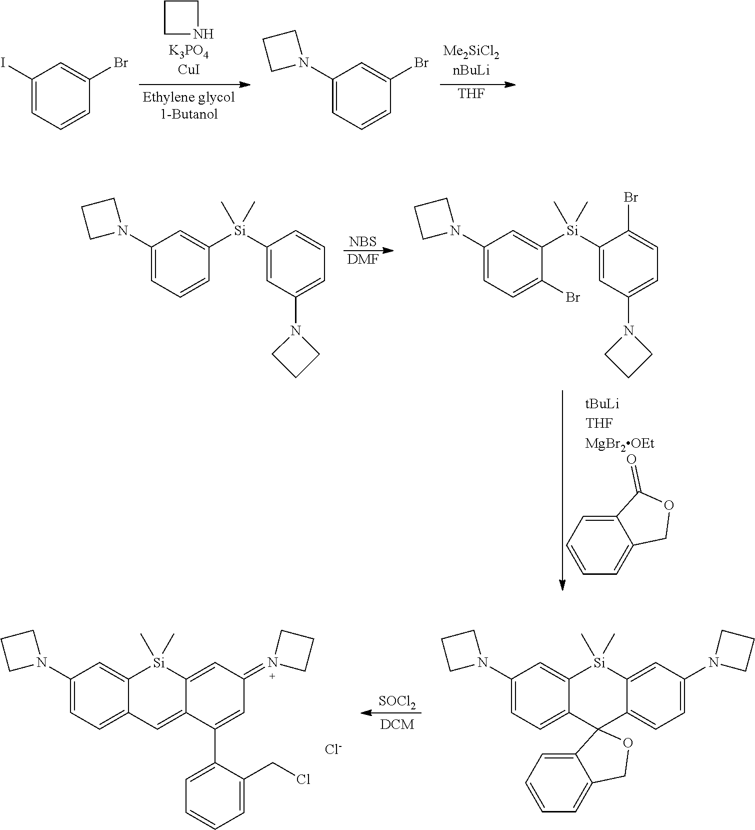

### Synthesis of 1-(3-bromophenyl)azetidine

3-Bromoiodobenzene (30 g, 106 mmol), azetidine (7.27 g, 127 mmol) and K₃PO₄ (67.5 g, 318 mmol) were combined with ethylene glycol (14.2 mL) and 1-butanol (150 mL) in a round bottom flask. The flask was sealed and evacuated/backfilled with nitrogen three times. CuI (2.02 g, 10.6 mmol) was subsequently added and the flask was again sealed and evacuated/backfilled with nitrogen three times. The mixture was then heated at 100° C. under an atmosphere of N₂ for 4 h. After cooling to room temperature, a saturated aqueous solution of NH₄Cl and EtOAc were added with stirring until there were no solids remaining. The layers were separated and the aqueous was extracted twice with EtOAc. The combined organic layers were washed with brine, then dried (MgSO₄) and filtered and the solvent was removed in vacuo. The residue was further dried under high vacuum. The crude product was purified by flash chromatography (0 to 10% Et₂O/PE) to give the title compound as a pale-yellow oil (18.2 g, 81%).

¹H NMR (CDCl₃, 400 MHz) δ 7.04 (1H, t), 6.85-6.80 (1H, m), 6.55 (1H, t), 6.36-6.31 (1H, m), 3.87 (4H, t), 2.37 (2H, p).

### Synthesis of bis(3-(azetidin-1-yl)phenyl)dimethylsilane

A solution of 1-(3-bromophenyl)azetidine (12.6 g, 59.5 mmol) in THF (115 mL) was cooled to −78° C. under nitrogen. A solution of n-butyllithium in hexane (2.5 M, 23.8 mL, 59.5 mmol) was slowly added so that the internal temperature was maintained below −60° C. during the addition. The reaction mixture was subsequently stirred at −78° C. for 30 min. A solution of dichlorodimethylsilane (3.20 g, 24.8 mmol) in THF (10 mL) was then added at a rate such that the internal temperature was kept below −60° C. The cooling bath was removed, and the reaction was stirred at room temperature for 3 h. It was subsequently quenched with saturated aqueous NH₄Cl (20 mL), diluted with water, and extracted twice with EtOAc. The combined organic extracts were washed with brine, dried over anhydrous MgSO₄, filtered, and concentrated in vacuo. The resulting residue was co-evaporated twice with Et₂O and purified by flash chromatography (o to 30% Et₂O/PE) to give the title compound as a colourless oil (8.00 g, 84%).

¹H NMR (CDCl₃, 400 MHz) δ 7.20 (2H, t), 6.90 (2H, d), 6.61 (2H, d), 6.46 (2H, ddd), 3.86 (8H, t), 2.34 (4H, p), 0.51 (6H, s).

### Synthesis of bis(5-(azetidin-1-yl)-2-bromophenyl)dimethylsilane

N-Bromosuccinimide (7-45 g, 41.9 mmol) was added in portions over 5 minutes to a solution of bis(3-(azetidin-1-yl)phenyl)dimethylsilane (6.75 g, 20.9 mmol) in DMF (120 mL). The resulting mixture was stirred for 5 days. Following removal of the solvent in vacuo, the resulting residue was diluted with water and extracted with EtOAc and then with DCM. The combined organic layers were washed with water and brine, then dried (MgSO₄) and filtered and the solvent was removed in vacuo. The crude product was purified by recrystallisation from EtOAc to give the title compound as a white solid (5.69 g, 57%).

¹H NMR (CDCl₃, 400 MHz) δ 7.31 (2H, d), 6.51 (2H, d), 6.31 (2H, dd), 3.81 (8H, t), 2.36 (4H, p), 0.71 (6H, s).

### Synthesis of 1,1′-(5,5-dimethyl-3′H,5H-spiro[dibenzo[b,e]siline-10,1′-isobenzofuran]-3,7-diyl)bis(azetidine)

A solution of t-BuLi in pentane (1.7 M, 10.3 mL) was added dropwise to a cooled (−78° C.) solution of bis(5-(azetidin-1-yl)-2-bromophenyl)dimethylsilane (2.00 g, 4.16 mmol) in THF (180 mL). After stirring for 20 minutes, the reaction mixture was warmed to −10° C. and a solution of MgBr₂·OEt₂ (2.37 g, 9.18 mmol) in THF (40 mL) was slowly added, keeping the internal temperature below −5° C. The reaction mixture was stirred at −10° C. for 30 minutes and then a solution of phthalide (1.23 g, 9.17 mmol) in THF (25 mL) was added dropwise over 30 minutes. The reaction mixture was allowed to warm to room temperature and was stirred for 60 h. Saturated aqueous NH₄Cl and EtOAc were added and the layers were separated, the aqueous was further extracted twice with EtOAc. The combined organic layers were washed with saturated aqueous NaHCO₃ and brine, dried over anhydrous MgSO₄, filtered, and concentrated in vacuo. The crude product was purified by flash chromatography (PE to 50% EtOAc/PE to a flush with 50% DCM/EtOAc) to give the title compound as an off-white solid (0.25 g, 14%).

¹H NMR (CDCl₃, 400 MHz) δ 7.31-7.21 (3H, m), 7.06 (1H, d), 6.95 (2H, d), 6.67 (2H, d), 6.31 (2H, dd), 5.21 (2H, s), 3.87 (8H, t), 2.34 (4H, p), 0.59 (3H, s), 0.52 (3H, s).

### Synthesis of 1-(7-(azetidin-1-yl)-10-(2-(chloromethyl)phenyl)-5,5-dimethyldibenzo[b,e]silin-3(5H)-ylidene)azetidin-1-ium chloride

Thionyl chloride (0.06 g, 0.50 mmol) was added dropwise to a solution of 1,1′-(5,5-dimethyl-3′H,5H-spiro[dibenzo[b,e]siline-10,1′-isobenzofuran]-3,7-diyl)bis(azetidine) (0.20 g, 0.46 mmol) in DCM (8 mL). After 20 minutes, the reaction mixture was diluted with water and DCM and the layers were separated. The aqueous layer was extracted twice with DCM and the combined organic layers were dried over anhydrous MgSO₄, filtered, and concentrated in vacuo. The crude product was purified by flash chromatography (5 to 20% MeOH/DCM) followed by precipitation from DCM/EtOAc to give the title compound as a blue/red solid (0.135 g, 60%).

¹H NMR (CDCl₃, 400 MHz) δ 7.63-7.61 (1H, m), 7.57-7.52 (1H, m), 7.49-7.45 (1H, m), 7.13 (1H, d), 6.93 (2H, d), 6.89 (2H, d), 6.26 (2H, dd), 4.51-4.36 (8H, m), 4.29 (2H, s), 2.60 (4H, p), 0.63 (3H, s), 0.60 (3H, s).

HRMS (ESI) calcd for C₂₈H₃₀ClN₂Si [M]⁺, 457.1867, found 457.1864.

### Example 2—1-(7-(azetidin-1-yl)-10-(4-(chloromethyl)phenyl)-5,5-dimethyldibenzo[b,e]silin-3(5H)-ylidene)azetidin-1-ium chloride

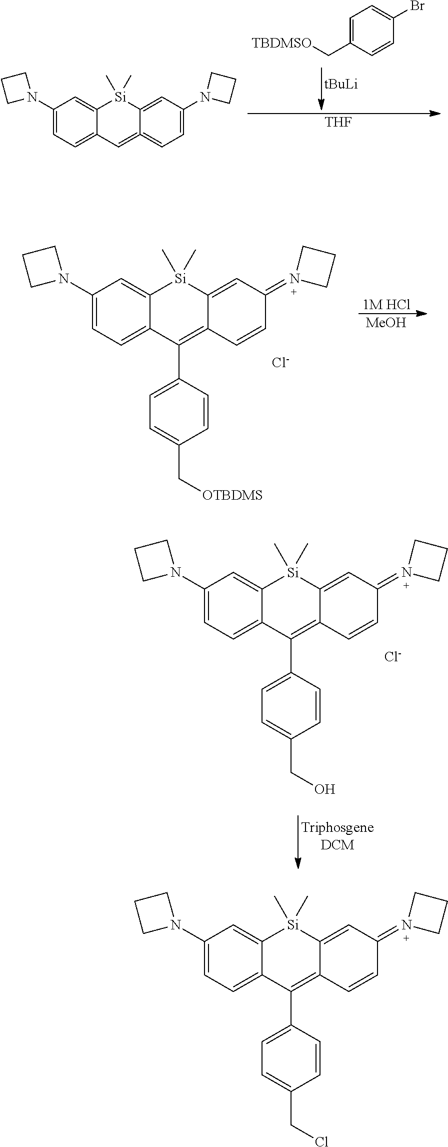

### Synthesis of 1-(7-(azetidin-1-yl)-10-(4-(((tert-butyldimethylsilyl)oxy)methyl)phenyl)-5,5-dimethyldibenzo[b,e]silin-3(5H)-ylidene)azetidin-1-ium chloride

A solution of t-BuLi in pentane (1.7 M, 2.49 mL) was added dropwise to a cooled (−78° C.) solution of (4-bromophenyl)methoxy-tert-butyldimethylsilane (0.60 g, 2.00 mmol) in THF (8 mL). After stirring for 10 minutes, a portion of this solution (5 mL) was slowly added to a suspension of 3,7-bis(azetidin-1-yl)-5,5-dimethyl-benzo[b][1]benzosilin-10-one (0.58 g, 1.66 mmol) in THF (12.5 mL). The resulting mixture was stirred overnight and was then diluted with both DCM and a saturated aqueous solution of NH₄Cl. The layers were separated and the aqueous was extracted three times with additional DCM. The combined organic layers were dried over anhydrous MgSO₄, filtered, and concentrated in vacuo. The resulting residue was dissolved in DCM and 5 drops of 2M aqueous HCl were added. The mixture was subsequently concentrated in vacuo and then co-evaporated with firstly MeOH and then with DCM. The crude product was purified by flash chromatography (5 to 20% MeOH in DCM) to give the title compound as a blue/green solid (0.14 g, 12%).

¹H NMR (CDCl₃, 400 MHz) δ 7.47-7.437.46 (2H, dm), 7.18-7.13 (2H, m), 7.07 (2H, d), 6.87 (2H, d), 6.23 (2H, dd), 4.86 (2H, s) 4.55-4.32 (8H, m), 2.61 (4H, p), 0.99 (9H, s), 0.60 (6H, s), 0.18 (6H, s).

LC/MS (ES+): m/z 553.4 (100%, M⁺).

### Synthesis of 1-(7-(azetidin-1-yl)-10-(4-(hydroxymethyl)phenyl)-5,5-dimethyldibenzo[b,e]silin-3(5H)-ylidene)azetidin-1-ium chloride

A 1M aqueous solution of HCl (0.4 mL) was added to a solution of 1-(7-(azetidin-1-yl)-10-(4-(((tert-butyldimethylsilyl)oxy)methyl)phenyl)-5,5-dimethyldibenzo[b,e]silin-3(5H)-ylidene)azetidin-1-ium chloride (0.056 g, 0.095 mmol) in MeOH (8 mL). After stirring for 30 minutes, the reaction mixture was diluted with DCM and water and the layers were separated. The aqueous was further extracted twice with DCM. The combined organic layers were dried over anhydrous MgSO₄, filtered, and concentrated in vacuo. The crude product was purified by flash chromatography (5 to 25% MeOH in DCM) to give the title compound as a blue/green solid (0.031 g, 71%).

¹H NMR (CDCl₃, 400 MHz) δ 7.59-7.55 (2H, m), 7.18-7.14 (2H, m), 7.12 (2H, d), 6.80 (2H, d), 6.26 (2H, dd), 4.87 (2H, s) 4.50-4.33 (8H, m), 3.03 (1H, s), 2.61 (4H, p), 0.58 (6H, s).

LC/MS (ES+): m/z 439.2 (100%, M⁺).

### Synthesis of 1-(7-(azetidin-1-yl)-10-(4-(chloromethyl)phenyl)-5,5-dimethyldibenzo[b,e]silin-3(5H)-ylidene)azetidin-1-ium chloride

A solution of triphosgene (0.008 g, 0.027 mmol) in DCM (0.5 mL) was added to a solution of 1-(7-(azetidin-1-yl)-10-(4-(hydroxymethyl)phenyl)-5,5-dimethyldibenzo[b,e]silin-3(5H)-ylidene)azetidin-1-ium chloride (0.021 g, 0.044 mmol) in DCM (4 mL). The reaction was monitored by LC/MS and after 30 minutes additional triphosgene/pyridine were added to push the reaction to completion. The mixture was diluted with DCM and washed with a 1M aqueous solution of HCl. The combined organic layers were dried over anhydrous MgSO₄, filtered, and concentrated in vacuo. The crude product was purified by flash chromatography (5 to 30% MeOH in DCM) followed by trituration with EtOAc to give the title compound as a blue/green solid (0.08 g, 37%).

¹H NMR (CDCl₃, 400 MHz) δ 7.57-7.51 (2H, m), 7.24-7.18 (2H, m), 7.02 (2H, d), 6.88 (2H, d), 6.28 (2H, dd), 4.70 (2H, s) 4.52-4.34 (8H, m), 2.62 (4H, p), 0.60 (6H, s).

LC/MS (ES+): m/z 457.3 (100%, M⁺).

### Example 3—1-(6-(azetidin-1-yl)-9-(2-(chloromethyl)phenyl)-3H-xanthen-3-ylidene)azetidin-1-ium chloride

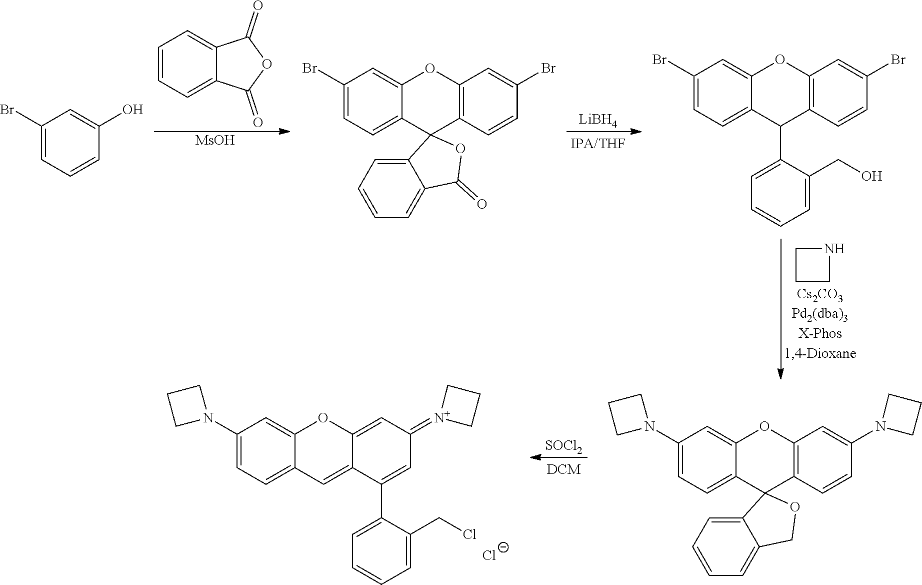

### Synthesis of 2-(3,6-dibromo-9H-xanthen-9-yl)benzoic acid

A mixture of phthalic anhydride (3.80 g, 25.7 mmol), 3-bromophenol (8.66 g, 50.0 mmol) and methanesulfonic acid (12.5 mL) was heated at 130° C. for 72 h. After cooling to room temperature, the reaction mixture was poured onto H₂O (700 mL) resulting in the formation of a precipitate. The precipitate was collected by filtration, washing with H₂O. The solid was subsequently dissolved in DCM (700 mL) with gentle heating and the solution was dried (MgSO₄), filtered and solvent was removed in vacuo. The crude product was heated with EtOAc (100 mL) allowed to cool and the solid was collected by filtration, washing with EtOAc to afford the title compound as a pale pink solid (4.20 g, 36%).

¹H NMR (CDCl₃, 400 MHz) δ 8.06-8.02 (1H, m), 7.71-7.62 (2H, m), 7.50 (2H, d), 7.19 (2H, dd), 7.14-7.10 (1H, m), 6.70 (2H, d).

LC/MS (ES+): m/z 458.9 (100%, [M+H]⁺).

### Synthesis of (2-(3,6-dibromo-9H-xanthen-9-yl)phenyl)methanol

A solution of LiBH₄ in THF (2M, 36.6 mL) was added dropwise to a cooled (o ° C.) suspension of 2-(3,6-dibromo-9H-xanthen-9-yl)benzoic acid (2.68 g, 5.85 mmol) in a mixture of THF (50 mL) and isopropanol (250 mL). The reaction mixture was allowed to warm to room temperature and stirred for 48 h. Subsequently, the reaction mixture was quenched by the cautious addition of saturated aqueous NH₄Cl (100 mL) and the product was extracted three times with DCM. The combined organic layers were dried (MgSO₄) and filtered and the solvent was removed in vacuo. The crude product was purified by flash chromatography (5 to 10% MeOH in DCM) to give the title compound (2.36 g, 90%) as a white solid.

¹H NMR (d6-DMSO, 400 MHz) δ 8.20 (1H, dd), 7.53-7.46 (3H, m), 7.44-7.32 (2H, m), 7.25 (2H, dd), 6.86 (2H, d), 6.77 (1H, s), 4.86 (1H, t), 3.46 (2H, d).

### Synthesis of 3′,6′-di(azetidin-1-yl)-3H-spiro[isobenzofuran-1,9′-xanthene]

A mixture of (2-(3,6-dibromo-9H-xanthen-9-yl)phenyl)methanol (2.36 g, 5.29 mmol), Pd₂(dba)₃ (0.484 g, 0.529 mmol), XPhos (0.69 g, 1.59 mmol) and Cs₂CO₃ (8.27 g, 25.4 mmol) in a round bottom flask was evacuated/backfilled with nitrogen (X3). To this mixture was added azetidine (0.664 g, 11.6 mmol) and 1,4-dioxane (40 mL) and the flask was again evacuated/backfilled three times with nitrogen. The flask was then inserted into a pre-heated metal heating block and stirred at 105° C. overnight. After cooling to room temperature, the reaction mixture was diluted with DCM/water and the layers were separated. The aqueous layer was extracted twice with additional DCM. The combined organic layers were dried (MgSO₄), filtered and the solvent was removed in vacuo. The crude product was purified by flash chromatography (2 to 15% MeOH in DCM) followed by trituration with EtOAc to give the title compound as a pale pink solid (0.62 g, 30%).

¹H NMR (d6-DMSO, 400 MHz) δ 7.46-7.39 (1H, m), 7.34 (1H, t), 7.23 (1H, t), 6.73 (1H, d), 6.65 (2H, d), 6.15-6.10 (4H, m), 5.16 (2H, s), 3.80 (8H, t), 2.29 (4H, p).

LC/MS (ES+): m/z 397.2 (100%, [M+H]⁺).

### Synthesis of 1-(6-(azetidin-1-yl)-9-(2-(chloromethyl)phenyl)-3H-xanthen-3-ylidene)azetidin-1-ium chloride

A stock solution of thionyl chloride (300 mg) in DCM (5 mL) was prepared. A portion (0.5 mL) of this stock solution was added dropwise to a solution of 3′,6′-di(azetidin-1-yl)-3H-spiro[isobenzofuran-1,9′-xanthene] (0.10 g, 0.252 mol) in DCM (10 mL). After 30 minutes, the reaction mixture was diluted with water and DCM and the layers were separated. The aqueous layer was extracted twice with DCM and the combined organic layers were dried over anhydrous MgSO₄, filtered, and concentrated in vacuo. The crude product was purified by flash chromatography (6 to 15% MeOH/DCM) followed by trituration with Et₂O to give the title compound as a dark red solid (0.114 g, 26%).

¹H NMR (CDCl₃, 400 MHz) δ 7.70-7.65 (1H, m), 7.62 (1H, t), 7.54 (1H, t), 7.19 (1H, d), 7.06-6.99 (2H, m), 6.58 (2H, dd), 6.49 (2H, d), 4.37 (8H, t), 4.26 (2H, s), 2.60 (4H, p).

HRMS (ESI) calcd for C₂₆H₂₄ClN₂O [M]⁺, 415.1577, found 415.1575.

### Example 4—1-(9-(2-(Chloromethyl)phenyl)-6-(3-methoxyazetidin-1-yl)-3H-xanthen-3-ylidene)-3-methoxyazetidin-1-ium chloride

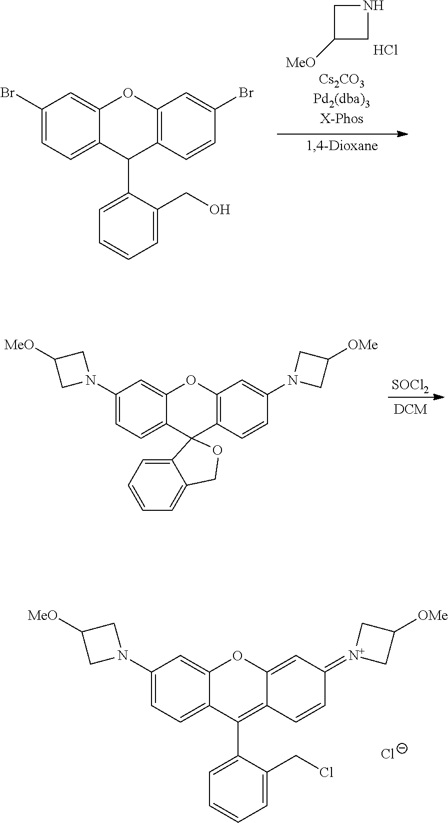

### Synthesis of 3′,6′-bis(3-methoxyazetidin-1-yl)-3H-spiro[isobenzofuran-1,9′-xanthene]

A mixture of (2-(3,6-dibromo-9H-xanthen-9-yl)phenyl)methanol (1.10 g, 2.47 mmol), Pd₂(dba)₃ (0.226 g, 0.247 mmol), XPhos (0.321 g, 0.74 mmol) and Cs₂CO₃ (3.86 g, 11.8 mmol) in a round bottom flask was evacuated/backfilled with nitrogen (X3). To this mixture was added 3-methoxyazetidine hydrochloride (0.67 g, 5.42 mmol) and 1,4-dioxane (20 mL) and the flask was again evacuated/backfilled three times with nitrogen. The flask was then inserted into a pre-heated metal heating block and stirred at 105° C. overnight. After cooling to room temperature, the reaction mixture was diluted with DCM/water and the layers were separated. The aqueous layer was extracted twice with additional DCM. The combined organic layers were dried (MgSO₄), filtered and the solvent was removed in vacuo. The crude product was purified by flash chromatography (4 to 15% MeOH in DCM) to give the title compound as a grey solid (0.32 g, 28%).

¹H NMR (CDCl₃, 400 MHz) δ 7.41-7.34 (2H, m), 7.32-7.24 (2H, m), 6.94 (1H, d), 6.77 (2H, d), 6.25 (1H, d), 6.17 (2H, dd), 5.26 (2H, s), 4.39-4.31 (2H, m), 4.11 (4H, t), 3.79-3.71 (4H, m), 3.35 (6H, s).

LC/MS (ES+): m/z 457.2 (100%, [M+H]⁺).

### Synthesis of 1-(9-(2-(chloromethyl)phenyl)-6-(3-methoxyazetidin-1-yl)-3H-xanthen-3-ylidene)-3-methoxyazetidin-1-ium chloride

A stock solution of thionyl chloride (235 mg) in DCM (10 mL) was prepared. A portion (1.0 mL) of this stock solution was added dropwise to a solution of 3′,6′-bis(3-methoxyazetidin-1-yl)-3H-spiro[isobenzofuran-1,9′-xanthene] (0.10 g, 0.219 mol) in DCM (10 mL). After 20 minutes, the reaction mixture was diluted with water and DCM and the layers were separated. The aqueous layer was extracted twice with DCM and the combined organic layers were dried over anhydrous MgSO₄, filtered, and concentrated in vacuo. The crude product was purified by flash chromatography (7 to 15% MeOH/DCM) followed by trituration with Et₂O to give the title compound as a dark purple solid (0.086 g, 77%).

¹H NMR (CDCl₃, 400 MHz) δ 7.70-7.59 (2H, m), 7.58-7.51 (1H, m), 7.19 (1H, d), 7.09-7.03 (2H, m), 6.64-6.55 (4H, m), 4.67-4.58 (4H, m), 4.56-4.49 (2H, m), 4.25 (2H, s), 4.17-4.11 (4H, m), 3.38 (6H, s).

HRMS (ESI) calcd for C₂₈H₂₈ClN₂O₃ [M]⁺, 475.1788, found 475.1791.

### REFERENCES

1) Grimm et al., General Synthetic Method for Si-Fluoresceins and Si-Rhodamines. ACS Cent Sci. 2017; 3(9):975-985.
2) Macho et al., Chloromethyl-X-rosamine is an aldehyde-fixable potential-sensitive fluorochrome for the detection of early apoptosis. Cytometry. 1996; 25(4): 333-340.
3) Poot et al.; Analysis of mitochondrial morphology and function with novel fixable fluorescent stains. J Histochem Cytochem. 1996; 4 4 (12): 1363-72.
4) Grimm et al., A general method to improve fluorophores for live-cell and single-molecule microscopy; Nat Methods. 2015 12(3): 244-50.
6) EP3 126451
7) U.S. Pat. No. 5,686,261

All publications mentioned in the above specification are herein incorporated by reference. Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A compound comprising a cationic species of formula (I): 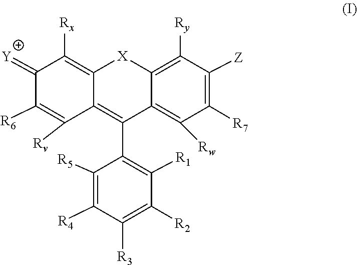
or a solvate, or tautomer thereof; and a counter ion;
wherein:
Y is a substituted or unsubstituted azetidine ring and Z is selected from OR₁₇ or a substituted or unsubstituted azetidine ring;
X is selected from O, S, SO₂, Se, NR₁₂, P(O)R₁₂, CR₁₃R₁₄, SiR₁₃R₁₄, Te, and GeR₁₃R₁₄;
R₁, R₂, R₃, R₄, and R₅ are each selected from H, C₁ to C₈ alkyl, OR₁₅, C(O)OR₁₆, NHC(O)R₁₅, C(O)NHR₁₅, halo, and a group of formula Q, wherein at least one of R₁, R₂, R₃, R₄, and R₅ is a group of formula Q;
Rᵥ, R_{w}, Rₓ, R_{y}, R₆, R₇ are each independently selected from H, C₁ to C₈ alkyl and halo;
R₁₆ is selected from C₁ to C₈ alkyl, optionally substituted aryl or optionally substituted heteroaryl;
R₁₇ is selected from H, C₁ to C₈ alkyl, optionally substituted aryl or optionally substituted heteroaryl; and
Q is a group comprising L-M_{A},
wherein M_{A} is selected from —CR₁₈R₁₉M_{B}, NHC(O)CR₁₈R₁₉M_{B}, C(O)NHCR₁₈R₁₉M_{B}, NHC(O)-L-CR₁₈R₁₉M_{B}, —C(O)NH-L-CR₁₈R₁₉M_{B}, —O—CR₁₈R₁₉M_{B} and —O-L-CR₁₈R₁₉M_{B};
each M_{B} is independently selected from halo, maleimidyl, OSO₂R₁₃, and 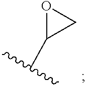 [SMILES: *C1CO1]
each L is an independently selected divalent linker group, substituted or unsubstituted phenylene or is absent, and
R₁₈ and R₁₉; are each independently selected from H and CH₃; and
R₁₂, R₁₃, R₁₄, and R₁₅ are each independently selected from H, C₁ to C₈ alkyl, optionally substituted aryl or optionally substituted heteroaryl.

2. A compound as claimed in claim 1, wherein at least one of Y and Z is a substituted or unsubstituted azetidine group of formula: 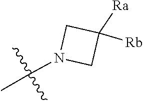
wherein R_{A} and R_{B} are independently selected from H, C₁ to C₈ alkyl, OR₂₀, C(O)OR₂₀, NHC(O)R₂₀, C(O)NHR₂₀, halo, NR₂₀R₂₁, —CN, —NC, optionally substituted aryl or optionally substituted heteroaryl; wherein R₂₀ and R₂₁ are independently selected from H, and C₁ to C₈ alkyl.

3. A compound as claimed in claim 1, wherein the cationic species is of formula (II): 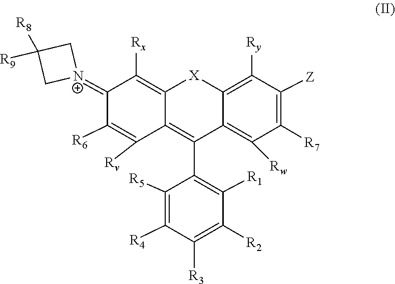
wherein R₈ and R₉ are independently selected from H, C₁ to C₈ alkyl, OR₂₀, C(O)OR₂₀, NHC(O)R₂₀, C(O)NHR₂₀, halo, NR₂₀R₂₁, —CN, —NC, optionally substituted aryl or optionally substituted heteroaryl.

4. A compound as claimed in claim 1, wherein the counter ion is a biologically compatible counter ion.

5. A compound as claimed in claim 1, wherein the counter ion is selected from halide, carboxylate, oxalate, sulfate, alkanesulfonate, arylsulfonate, phosphate, perchlorate, tetrafluoroborate, tetraphenylboride, hexafluorophosphate, nitrate and anions of aromatic or aliphatic carboxylic acids.

6. A method as claimed in claim 1, wherein the cationic species is of formula (III): 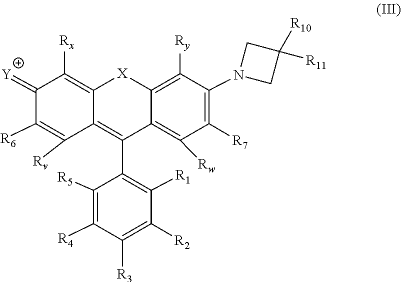
wherein R₁₀ and R₁₁ are independently selected from H, C₁ to C₈ alkyl, OR₂₀, C(O)OR₂₀, NHC(O)R₂₀, C(O)NHR₂₀, halo, NR₂₀R₂₁, —CN, —NC, optionally substituted aryl or optionally substituted heteroaryl.

7. A compound as claimed in claim 1, wherein the cationic species is of formula (IV):

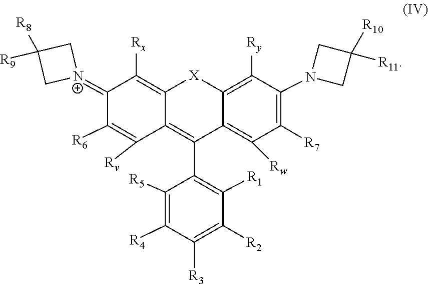

8. A compound as claimed in claim 1, wherein at least one of R₁ and R₅ is Q.

9. A compound as claimed in claim 1, wherein R₃ is Q.

10. A compound as claimed in claim 1, wherein at least one of R₂ and R₄ is Q.

11. A compound as claimed in claim 1, wherein L comprises an alkylene chain —(CH₂)ₘ—, wherein m is 1 to 6.

12. A compound as claimed in claim 1, wherein M_{B} is halo.

13. A compound as claimed in claim 10, wherein M_{B} is chloro.

14. A compound as claimed in claim 1, wherein the compound comprises a cationic species selected from species of formulae:

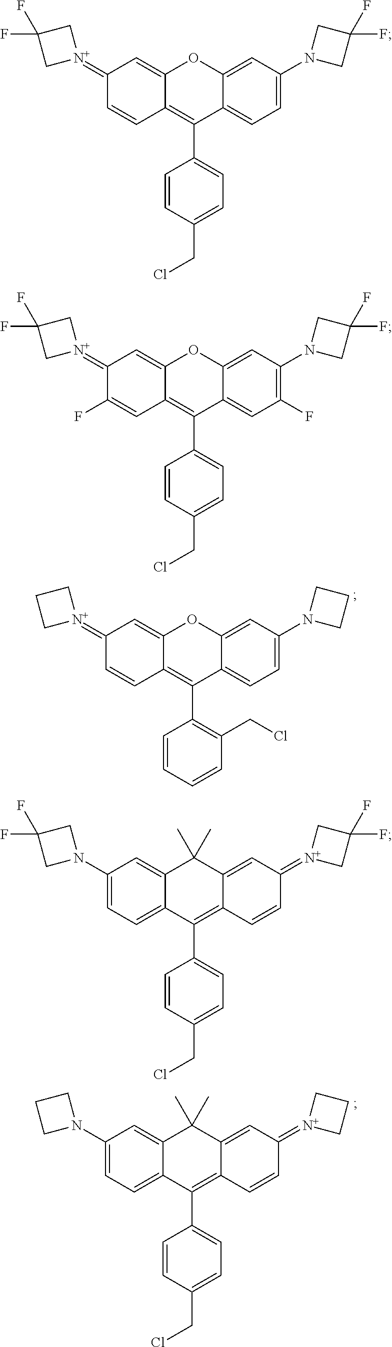 [SMILES: FC1(F)CN(c2ccc3c(-c4ccc(CCl)cc4)c4ccc(=[N+]5CC(F)(F)C5)cc-4oc3c2)C1 ; Fc1cc2c(-c3ccc(CCl)cc3)c3cc(F)c(=[N+]4CC(F)(F)C4)cc-3oc2cc1N1CC(F)(F)C1 ; ClCc1ccccc1-c1c2ccc(=[N+]3CCC3)cc-2oc2cc(N3CCC3)ccc12 ; CC1(C)C2=CC(=[N+]3CC(F)(F)C3)C=CC2=C(c2ccc(CCl)cc2)c2ccc(N3CC(F)(F)C3)cc21 ; CC1(C)C2=CC(=[N+]3CCC3)C=CC2=C(c2ccc(CCl)cc2)c2ccc(N3CCC3)cc21]

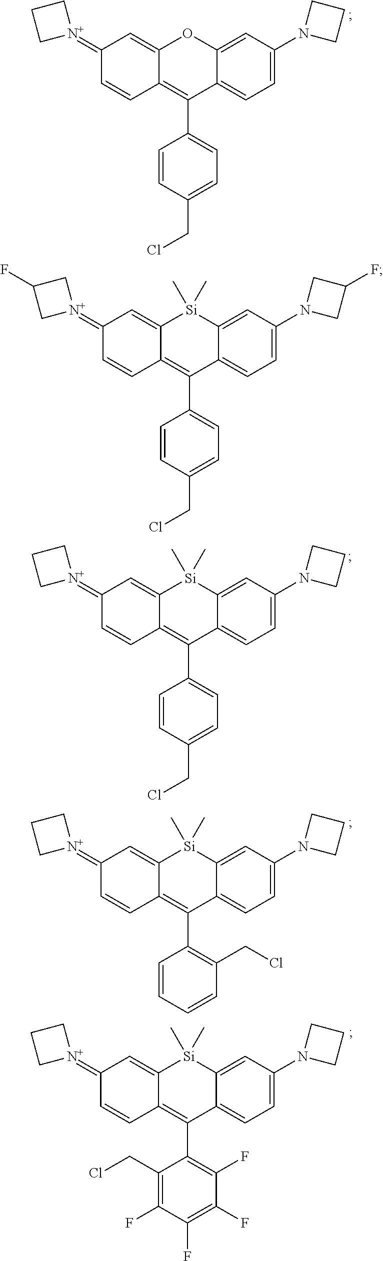 [SMILES: ClCc1ccc(-c2c3ccc(=[N+]4CCC4)cc-3oc3cc(N4CCC4)ccc23)cc1 ; C[Si]1(C)C2=CC(=[N+]3CC(F)C3)C=CC2=C(c2ccc(CCl)cc2)c2ccc(N3CC(F)C3)cc21 ; C[Si]1(C)C2=CC(=[N+]3CCC3)C=CC2=C(c2ccc(CCl)cc2)c2ccc(N3CCC3)cc21 ; C[Si]1(C)C2=CC(=[N+]3CCC3)C=CC2=C(c2ccccc2CCl)c2ccc(N3CCC3)cc21 ; C[Si]1(C)C2=CC(=[N+]3CCC3)C=CC2=C(c2c(F)c(F)c(F)c(F)c2CCl)c2ccc(N3CCC3)cc21]

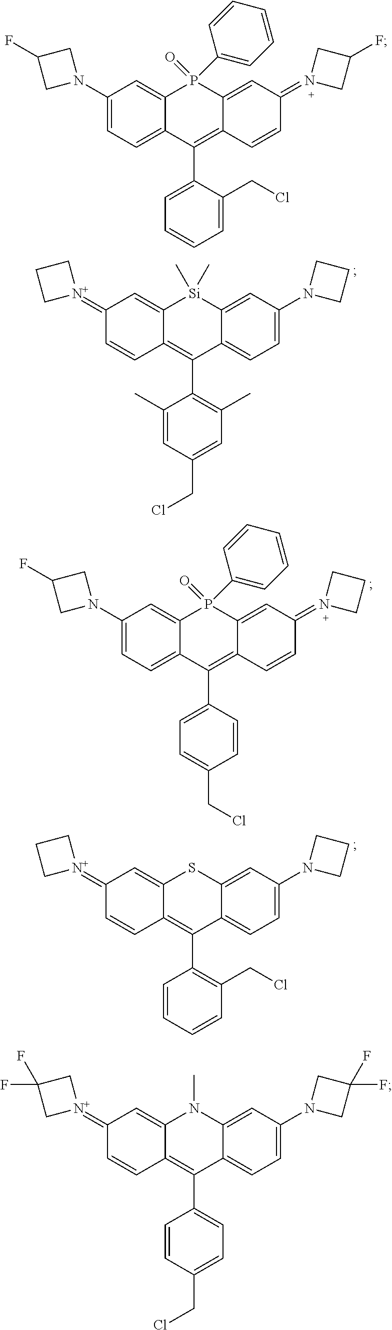

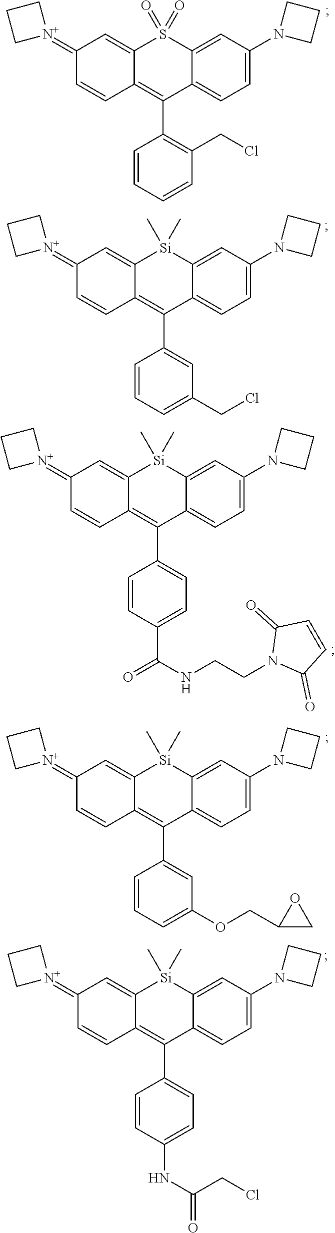 [SMILES: O=S1(=O)C2=CC(=[N+]3CCC3)C=CC2=C(c2ccccc2CCl)c2ccc(N3CCC3)cc21 ; C[Si]1(C)C2=CC(=[N+]3CCC3)C=CC2=C(c2cccc(CCl)c2)c2ccc(N3CCC3)cc21 ; C[Si]1(C)C2=CC(=[N+]3CCC3)C=CC2=C(c2ccc(C(=O)NCCN3C(=O)C=CC3=O)cc2)c2ccc(N3CCC3)cc21 ; C[Si]1(C)C2=CC(=[N+]3CCC3)C=CC2=C(c2cccc(OCC3CO3)c2)c2ccc(N3CCC3)cc21 ; C[Si]1(C)C2=CC(=[N+]3CCC3)C=CC2=C(c2ccc(NC(=O)CCl)cc2)c2ccc(N3CCC3)cc21]

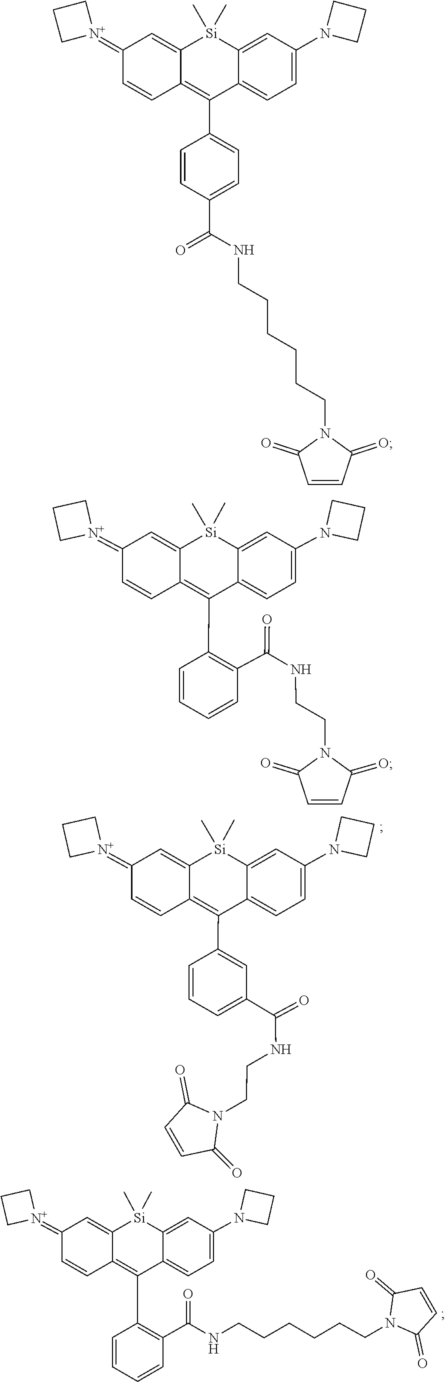 [SMILES: C[Si]1(C)C2=CC(=[N+]3CCC3)C=CC2=C(c2ccc(C(=O)NCCCCCCN3C(=O)C=CC3=O)cc2)c2ccc(N3CCC3)cc21 ; C[Si]1(C)C2=CC(=[N+]3CCC3)C=CC2=C(c2ccccc2C(=O)NCCN2C(=O)C=CC2=O)c2ccc(N3CCC3)cc21 ; C[Si]1(C)C2=CC(=[N+]3CCC3)C=CC2=C(c2cccc(C(=O)NCCN3C(=O)C=CC3=O)c2)c2ccc(N3CCC3)cc21 ; C[Si]1(C)C2=CC(=[N+]3CCC3)C=CC2=C(c2ccccc2C(=O)NCCCCCCN2C(=O)C=CC2=O)c2ccc(N3CCC3)cc21]

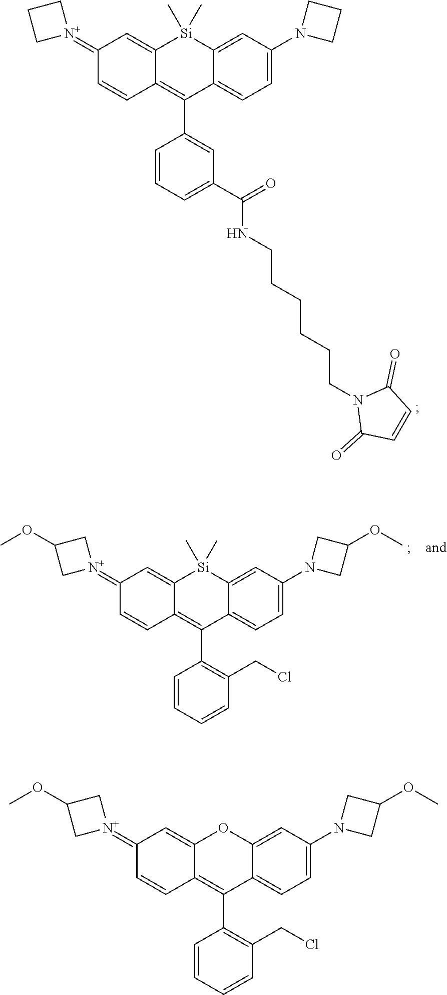

15.-18. (canceled)

19. A method for staining mitochondria, the method comprising:
providing a sample containing mitochondria, and
incubating the sample in a composition comprising a compound as claimed in claim 1.

20. A method as claimed in claim 19, wherein incubating the sample is for a predetermined time in the range 10 mins to 2 hours and at a predetermined temperature in the range 20° C. to 39° C.

21. A method as claimed in claim 19, wherein the sample containing mitochondria comprises a tissue sample.

22. A method as claimed in claim 19, wherein the sample containing mitochondria is a plant, animal or fungal tissue sample, a sample of plant, animal or fungal cells or isolated plant, animal or fungal mitochondria.

23. A method as claimed in claim 19, wherein the sample containing mitochondria comprises a sample containing fixed mitochondria and/or a sample containing mitochondria in fixed cells.

24. A method as claimed in claim 19, wherein the sample containing mitochondria contains substantially no live cells.

25. A method of analysing mitochondria, the method comprising:
staining a sample of mitochondria using a compound as claimed in claim 1,
fixing the cells,
illuminating the stained sample using light of an appropriate wavelength to fluoresce the compound, and
observing or imaging a magnified image of the sample.

26. A method as claimed in claim 25, wherein the appropriate wavelength is in the range 400 nm to 800 nm.

27. A method of detecting a mitochondrial condition, the method comprising staining a sample of mitochondria as claimed in claim 18.

28. A method as claimed in claim 27, wherein the sample of mitochondria is a plant, animal or fungal tissue sample, a sample of plant, animal or fungal cells or isolated plant, animal or fungal mitochondria.

29. A compound as claimed in claim 1, wherein each L is independently selected from C₁ to C₈ alkylene.
